SEQUENCE LISTING (8 sequences)

SEQ ID NO: 1 - DNA, 18 bp, Artificial Sequence
  Synthetic primer
tgggtaaaacctatcatc

SEQ ID NO: 2 - DNA, 15 bp, Artificial Sequence
  Synthetic primer
aagcccatcaccatt

SEQ ID NO: 3 - DNA, 17 bp, Artificial Sequence
  Synthetic primer
gatacgtagcatgtcat

SEQ ID NO: 4 - DNA, 13 bp, Artificial Sequence
  Synthetic primer
cgtcatcgtacac

SEQ ID NO: 5 - DNA, 28 bp, Artificial Sequence
  Synthetic primer
tcagagaagccattatctgcaaaaatat

SEQ ID NO: 6 - DNA, 22 bp, Artificial Sequence
  Synthetic primer
ggccatgaaaaaaaaaacatgc

SEQ ID NO: 7 - DNA, 21 bp, Artificial Sequence
  Synthetic primer
aaaacatcccacgcctagtcc

SEQ ID NO: 8 - DNA, 19 bp, Artificial Sequence
  Synthetic primer
gtgcccaggtcagtggatc

(19) United States
(12) Patent Application Publication
(10) Pub. No.: US 2013/0123335 A1
(43) Pub. Date: May 16, 2013

(54) IDH1 AND IDH2 MUTATIONS IN CHOLANGIOCARCINOMA

(71) Applicants: Benjamin R. Kipp, Rocheste, MN (US); Jesse S. Voss, Rochester, MN (US)

(72) Inventors: Benjamin R. Kipp, Rocheste, MN (US); Jesse S. Voss, Rochester, MN (US)

(73) Assignee: MAYO FOUNDATION FOR MEDICAL EDUCATION AND RESEARCH, Rochester, MN (US)

(21) Appl. No.: 13/606,662

(22) Filed: Sep. 7, 2012

Related U.S. Application Data
(60) Provisional application No. 61/533,636, filed on Sep. 12, 2011.

(51) Int. Cl. C12Q 1/68

(52) U.S. Cl. CPC C12Q 1/6886
USPC 514/44 A; 435/6.11

(57) ABSTRACT

This document relates to methods and materials involved in assessing isocitrate dehydrogenase 1 (IDH1) or isocitrate dehydrogenase 2 (IDH2) mutations in a mammal (e.g., human). For example, this document provides methods and materials for diagnosis, characterization, determining prognosis, and treatment of cholangiocarcinoma tumor in a mammal.

This application claims benefit of priority to U.S. Provisional Application Ser. No. 61/533,636, filed Sep. 12, 2011, the entire contents of which are hereby incorporated by reference.

### BACKGROUND OF THE INVENTION

1. Field of the Invention

This document relates to methods and materials involved in assessing isocitrate dehydrogenase 1 (IDH1) or isocitrate dehydrogenase 2 (IDH2) mutations in a mammal (e.g., human). For example, this document provides methods and materials for diagnosis of cholangiocarcinoma (CCA) by screening for the presence or absence of IDH1 and IDH2 mutations. For example, this document provides methods and materials for characterizing a CCA tumor sample by determining the presence or absence of IDH1 or IDH2 mutations in the sample. In some aspects, this document relates to determining the prognosis of a mammal having a CCA tumor, comprising determining the presence or absence of a mutation in IDH1 or IDH2 in a sample.

2. Description of the Related Art

Cholangiocarcinoma (CCA) is a tumor arising from malignant transformation of biliary tract epithelium. CCA's can present as an intrahepatic mass or an obstructing tumor involving the extrahepatic and/or intrahepatic bile ducts (Lazaridis et al., 2005; Blechacz et al., 2008). Curative treatments for early stage CCA include surgical resection or liver transplantation (Blechacz et al., 2008; Rosen et al., 2010; Akamatsu et al., 2011). Unfortunately, the median survival of patients with CCA is less than 24 months because most patients present with advanced stage disease, which is not amenable to surgical therapies (Blechacz et al., 2008; Rosen et al., 2010; Gatto et al., 2010).

There are various clinical tests used to diagnose CCA. Histopathology and cytopathology are often considered the gold standards for pathologic diagnosis. Currently, no single imaging method emerges for the diagnosis of cholangiocarcinoma. Among the imaging methods used are ultrasonography, computed tomography, magnetic resonance imaging, and cholangiography. As for tumor biomarkers, the most commonly used markers are carbohydrate antigen 19-9 (CA 19-9) and carcinoembryonic antigen (CEA). Studies by Levy et al. suggest that CA 19-9 only identifies patients with advanced, unresectable cholangiocarcinomas (Levy et al., 2005). Several authors have suggested that the diagnostic yield of CEA in the detection of cholangiocarcinoma is lower than that of CEA (Nehls et al., 2004). The combined use of CEA and CA 19-9 may improve the diagnosis of cholangiocarcinoma (Ramage et al., 1995), but this has not been reproduced in all studies (Bjornsson et al., 1999). Several other potential serum tumor biomarkers have been linked to cholangiocarcinoma including CA-195, CA-242, DU-PAN-2, IL-6, and trypsinogen-2, but their clinical role is currently unclear.

Thus, the diagnosis of cholangiocarcinoma remains difficult, despite the multiple diagnostic methods available. Recent advancements in imaging technologies and the clinical implementation of new molecular markers have modestly improved the ability to detect CCA earlier (Patel et al., 2011). Unfortunately, improved biomarkers are still needed to detect the majority of CCA's at eelier stages when treatments are beneficial.

### SUMMARY OF THE INVENTION

This document relates to methods and materials involved in assessing isocitrate dehydrogenase 1 (IDH1) or isocitrate dehydrogenase 2 (IDH2) mutations in a mammal (e.g., human). For example, this document provides methods and materials for diagnosis of cholangiocarcinoma (CCA) by screening for the presence or absence of IDH1 and IDH2 mutations. For example, this document provides methods and materials for characterizing a CCA tumor sample by determining the presence or absence of IDH1 or IDH2 mutations in the sample. In some aspects, this document relates to determining the prognosis of a mammal having a CCA tumor, comprising determining the presence or absence of a mutation in IDH1 or IDH2 in a sample.

As described herein, sequence analysis of 94 CCA specimens (67 intrahepatic and 27 extrahepatic) revealed 21 specimens with IDH mutations (19 intrahepatic and 2 extrahepatic) including 14 specimens with IDH1 mutations and 7 specimens with IDH2 mutations. The results provided herein show for the first time that IDH1 and IDH2 genes are mutated in CCA. This can allow physicians to develop a clinical assay to diagnose and characterize CCA tumors based on the presence or absence of IDH1 and IDH2 mutations, as well as to aid in prognosis and selection of a particular treatment for this condition.

In accordance with the present disclosure, there is provided a method of diagnosing a cholangiocarcinoma tumor of intrahepatic origin in a mammal comprising (a) performing a histologic analysis of a tumor cell-containing sample from said mammal, whereby glioma and secondary glioblastomas, acute myeloid leukemia, and chondrosarcoma are excluded by histology; and (b) sequencing a isocitrate dehydrogenase 1 (IDH1) or isocitrate dehydrogenase 2 (IDH2) encoding polynucleotide in from a tumor-cell containing sample from said mammal to identify the presence or absence of a mutation in IDH1 or IDH2, wherein the presence of a mutation in IDH1 or IDH2 excludes distal extrahepatic cholangiocarcinoma, thereby diagnosing a cholangiocarcinoma of intrahepatic origin. The mutation may be R132c in IDH1, R132S in IDH1, R132G in IDH1, R132L in IDH1, R172M in IDH2, R172K in IDH2, or R172G in IDH2. The mammal may be a human or a non-human mammal. The method may further comprise measuring 2-hydroxyglutarate in a tumor from said mammal.

In another embodiment, there is provided a method of treating a cholangiocarcinoma tumor in a mammal comprising (a) sequencing a isocitrate dehydrogenase 1 (IDH1) or isocitrate dehydrogenase 2 (IDH2) encoding polynucleotide in from tumor sample from said mammal to identify the presence or absence of a mutation in IDH1 or IDH2; (b) treating said mammal with an inhibitor of 2-hydroxyglutarate synthesis or function when a mutation in IDH1 or IDH2 is found. The mutation may be R132c in IDH1, R132S in IDH1, R132G in IDH1, R132L in IDH1, R172M in IDH2, R172K in IDH2, or R172G in IDH2. The mammal may be a human or a non-human mammal. The method may further comprise measuring 2-hydroxyglutarate in a tumor from said mammal.

In yet another embodiment, there is provided a method for predicting the survival of a mammal having a cholangiocarcinoma tumor comprising sequencing a isocitrate dehydrogenase 1 (IDH1) or isocitrate dehydrogenase 2 (IDH2) encoding polynucleotide in from tumor sample from said mammal to identify the presence or absence of a mutation in IDH1 or IDH2, whereby the presence of IDH1 and/or IDH2 mutation indicates better overall survival than the absence of IDH1 and/or IDH2 mutation. The mutation may be R132c in IDH1, R132S in IDH1, R132G in IDH1, R132L in IDH1, R172M in IDH2, R172K in IDH2, or R172G in IDH2. The mammal may be a human or a non-human mammal. The method may further comprise measuring 2-hydroxyglutarate in a tumor from said mammal.

Unless otherwise defined, all technical and scientific terms used herein have the same meaning as commonly understood by one of ordinary skill in the art to which this invention pertains All publications, patent applications, patents, and other references mentioned herein are incorporated by reference in their entirety. In case of conflict, the present specification, including definitions, will control. In addition, the materials, methods, and examples are illustrative only and not intended to be limiting.

Any embodiment discussed with respect to one aspect of the invention applies to other aspects of the invention as well, and the embodiments in the Examples section are understood to be embodiments of the invention that are applicable to all aspects of the invention.

The use of the term “or” in the claims is used to mean “and/or” unless explicitly indicated to refer to alternatives only or the alternatives are mutually exclusive, although the disclosure supports a definition that refers to only alternatives and “and/or.”

Throughout this application, the term “about” is used to indicate that a value includes the standard deviation of error for the device or method being employed to determine the value.

Following long-standing patent law, the words “a” and “an,” when used in conjunction with the word “comprising” in the claims or specification, denotes one or more, unless specifically noted.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE FIGURES

The following drawings form part of the present specification and are included to further demonstrate certain aspects of the present invention. The invention may be better understood by reference to one or more of these drawings in combination with the detailed description of specific embodiments presented herein.

FIG. 1. Representative example of the IDH1 (top) and IDH2 (bottom) mutations identified by Sanger sequencing (middle) and pyrosequencing (right).

FIG. 2. Survival of patients with cholangiocarcinoma with or without IDH1 or IDH2 gene mutations.

FIGS. 3A-B. Metastatic poorly differentiated cholangiocarcinoma. (FIG. 3A) forming sheets of malignant cells in a hepatoduodenal lymph node (4×); (FIG. 3B) The metastasis has a nested morphology (×20).

FIG. 4. Metastatic poorly differentiated cholangiocarcinoma associated with desmoplasia and prominent sclerosis (4×).

FIG. 5. Examples of wild-type and IDH1/2 mutant pyrosequencing and Sanger sequencing results from metastatic cholangiocarcinoma specimens

### DESCRIPTION OF THE ILLUSTRATIVE EMBODIMENTS

This document relates to methods and materials involved in assessing isocitrate dehydrogenase 1 (IDH1) or isocitrate dehydrogenase 2 (IDH2) mutations in a mammal (e.g., human). For example, this document provides methods and materials for diagnosis of cholangiocarcinoma (CCA) by screening for the presence or absence of IDH1 and IDH2 mutations. For example, this document provides methods and materials for characterizing a CCA tumor sample by determining the presence or absence of IDH1 or IDH2 mutations in the sample. In some aspects, this document relates to determining the prognosis of a mammal having a CCA tumor, comprising determining the presence or absence of a mutation in IDH1 or IDH2 in a sample.

The methods and materials provided herein can be used to diagnose CCA by screening for the presence or absence of IDH1 or IDH2 mutations in a sample from a mammal. In some cases, the methods and materials provided herein can be used to determine whether or not a sample from a mammal contains one or more IDH1 mutations. For example, the IDH1 mutation can be selected from the group consisting of R132c, R132S, R132G, and R132L. In some cases, the methods and materials provided herein can be used to determine whether or not a sample from a mammal contains one or more IDH2 mutations. For example, the IDH2 mutation can be selected from the group consisting of R172M, R172K and R172G.

In some cases, the presence or absence of an IDH1 or IDH2 mutation in a sample can be used to determine the prognosis of a mammal with CCA. For example, the presence of an IDH1 or IDH2 mutation in a CCA sample can indicate that the mammal has increased survival after surgery. For example, the absence of an IDH1 or IDH2 mutation in a CCA sample can indicate that the mammal has decreased survival after surgery.

In some cases, the presence or absence of an IDH1 or IDH2 mutation can be used to characterize a CCA tumor sample. The presence or absence of an IDH1 or IDH2 mutation in a CCA sample can be used to select an appropriate treatment for a mammal. For example, a mammal with a CCA tumor sample containing an IDH1 or IDH2 mutation can be treated with an agent that specifically targets IDH1 or IDH2. The agent can include a small molecule inhibitor, an antibody, siRNA, or an agent that targets 2-hydroxygluterate. For example, a mammal with a CCA tumor sample containing an IDH1 or IDH2 mutation can be assigned to a clinical trial group.

### 1. CHOLANGIOCARCINOMA

Cholangiocarcinoma is a medical term denoting a form of cancer that is composed of mutated epithelial cells (or cells showing characteristics of epithelial differentiation) that originate in the bile ducts which drain bile from the liver into the small intestine. Other biliary tract cancers include pancreatic cancer, gallbladder cancer, and cancer of the ampulla of Vater. Cholangiocarcinoma is a relatively rare neoplasm that is classified as an adenocarcinoma (a cancer that forms glands or secretes significant amounts of mucins). It has an annual incidence rate of 1-2 cases per 100,000 in the Western world, but rates of cholangiocarcinoma have been rising worldwide over the past several decades.

Prominent signs and symptoms of cholangiocarcinoma include abnormal liver function tests, abdominal pain, jaundice, and weight loss. generalized itching, fever, and changes in color of stool or urine may also occur. The disease is diagnosed through a combination of blood tests, imaging, endoscopy, and sometimes surgical exploration, with confirmation obtained after a pathologist examines cells from the tumor under a microscope. Known risk factors for cholangiocarcinoma include primary sclerosing cholangitis (an inflammatory disease of the bile ducts), congenital liver malformations, infection with the parasitic liver flukes Opisthorchis viverrini or Clonorchis sinensis, and exposure to Thorotrast (thorium dioxide), a chemical formerly used in medical imaging. However, most patients with cholangiocarcinoma have no identifiable specific risk factors.

Cholangiocarcinoma is considered to be an incurable and rapidly lethal malignancy unless both the primary tumor and any metastases can be fully resected (removed surgically). No potentially curative treatment yet exists except surgery, but most patients have advanced stage disease at presentation and are inoperable at the time of diagnosis. Patients with cholangiocarcinoma are generally managed—though never cured—with chemotherapy, radiation therapy, and other palliative care measures. These are also used as adjuvant therapies (i.e., post-surgically) in cases where resection has apparently been successful (or nearly so). Some areas of ongoing medical research in cholangiocarcinoma include the use of newer targeted therapies, (such as erlotinib) or photodynamic therapy for treatment, and the techniques to measure the concentration of byproducts of cancer stromal cell formation in the blood for diagnostic purposes.

Although there are at least three staging systems for cholangiocarcinoma (e.g., those of Bismuth, Blumgart, and the American Joint Committee on Cancer), none have been shown to be useful in predicting survival. The most important staging issue is whether the tumor can be surgically removed, or whether it is too advanced for surgical treatment to be successful. Often, this determination can only be made at the time of surgery. General guidelines for operability include:
Absence of lymph node or liver metastases
Absence of involvement of the portal vein
Absence of direct invasion of adjacent organs
Absence of widespread metastatic disease

The most common physical indications of cholangiocarcinoma are abnormal liver function tests, jaundice (yellowing of the eyes and skin occurring when bile ducts are blocked by tumor), abdominal pain (30%-50%), generalized itching (66%), weight loss (30%-50%), fever (up to 20%), and changes in stool or urine color. To some extent, the symptoms depend upon the location of the tumor: patients with cholangiocarcinoma in the extrahepatic bile ducts (outside the liver) are more likely to have jaundice, while those with tumors of the bile ducts within the liver more often have pain without jaundice.

Blood tests of liver function in patients with cholangiocarcinoma often reveal a so-called “obstructive picture,” with elevated bilirubin, alkaline phosphatase, and gamma glutamyl transferase levels, and relatively normal transaminase levels. Such laboratory findings suggest obstruction of the bile ducts, rather than inflammation or infection of the liver parenchyma, as the primary cause of the jaundice. CA19-9 is elevated in most cases of cholangiocarcinoma.

Cholangiocarcinoma can affect any area of the bile ducts, either within or outside the liver. Tumors occurring in the bile ducts within the liver are referred to as intrahepatic, those occurring in the ducts outside the liver are extrahepatic, and tumors occurring at the site where the bile ducts exit the liver may be referred to as perihilar. A cholangiocarcinoma occurring at the junction where the left and right hepatic ducts meet to form the common bile duct may be referred to eponymously as a Klatskin tumor.

Although cholangiocarcinoma is known have the histological and molecular features of an adenocarcinoma of epithelial cells lining the biliary tract, the actual cell of origin is unknown. Recent evidence has suggested that the initial transformed cell that generates the primary tumor may arise from a pluripotent hepatic stem cell. Cholangiocarcinoma is thought to develop through a series of stages—from early hyperplasia and metaplasia, through dysplasia, to the development of frank carcinoma—in a process similar to that seen in the development of colon cancer. Chronic inflammation and obstruction of the bile ducts, and the resulting impaired bile flow, are thought to play a role in this progression.

Histologically, cholangiocarcinomas may vary from undifferentiated to well-differentiated. They are often surrounded by a brisk fibrotic or desmoplastic tissue response; in the presence of extensive fibrosis, it can be difficult to distinguish well-differentiated cholangiocarcinoma from normal reactive epithelium. There is no entirely specific immunohistochemical stain that can distinguish malignant from benign biliary ductal tissue, although staining for cytokeratins, carcinoembryonic antigen, and mucins may aid in diagnosis. Most tumors (>90%) are adenocarcinomas.

### 2. SUBJECTS AND SAMPLES

A mammal can be any type of mammal including, without limitation, a mouse, rat, dog, cat, horse, sheep, goat, cow, pig, monkey, or human. In some cases, a sample can be obtained from a mammal suspected of having CCA. In some cases, a sample can be obtained from a mammal known to have CCA.

A sample can be any biological specimen useful for characterizing the presence of CCA in a sample. For example, specimens can include biliary tract brushings, bile aspirates, bile washings, fine needle aspirates, or tissue specimens from biliary tract or liver.

### 3. IDH1 And IDH2 ENZYMES

IDH1 and IDH2 are NADP⁺-dependent enzymes encoded by IDH1 and IDH2 genes, which catalyze the oxidative decarboxylation of isocitrate to α-ketogluterate (α-KG) (Yan et al., 2009; Watanabe et al., 2009; Tefferi et al., 2010; Sanson et al., 2009; Reitman et al., 2010; Ichimura et al., 2009; Hartmann et al., 2009; and Bleeker et al., 2009). Somatic mutations in IDH1 and IDH2 result in proteins with neomorphic enzyme activity that allows α-KG to be more effectively converted to 2-hydroxygluterate (2-HG) (Pietrak et al., 2011 and Dang et al., 2009). Increased levels of 2-HG are thought to promote carcinogenesis by competitively inhibiting enzymes that use α-KG as a cofactor (Pietrak et al., 2011; Dang et al., 2009; Ward et al., 2010; and Reitman et al., 2011).

### 4. Sample Analysis

The IDH1 or IDH2 gene, transcript, and protein may be detected in cultured cells or cells isolated from a mammal using any of the methods described in the instant application or those well known in the art. In some cases, the presence or absence of an IDH1 or IDH2 mutation can be detected by assessing the gene sequence or transcript of the gene. For example, an IDH1 and IDH2 gene may be detected by Southern blot, PCR, sequencing, a peptide nucleic acid-locked nucleic acid clamp method, Northern blot, RT-PCR, and the like. In some cases, the presence or absence of an IDH1 or IDH2 mutation can be detected by assessing the protein sequence, expression levels and/or distribution.

For example an IDH1 and IDH2 protein may be detected by immunohistochemistry, Western blot, mass spectrometry, and the like. These techniques are discussed in greater detail below.

A. Protein-Based Detection—Immunodetection

There are a variety of methods that can be used to assess protein expression. One such approach is to perform protein identification with the use of antibodies. As used herein, the term “antibody” is intended to refer broadly to any immunologic binding agent such as IgG, IgM, IgA, IgD and IgE. Generally, IgG and/or IgM are preferred because they are the most common antibodies in the physiological situation and because they are most easily made in a laboratory setting. The term “antibody” also refers to any antibody-like molecule that has an antigen binding region, and includes antibody fragments such as Fab′, Fab, F(ab′)₂, single domain antibodies (DABs), Fv, scFv (single chain Fv), and the like. The techniques for preparing and using various antibody-based constructs and fragments are well known in the art. Means for preparing and characterizing antibodies, both polyclonal and monoclonal, are also well known in the art (See, e.g., Antibodies: A Laboratory Manual, Cold Spring Harbor Laboratory, 1988; incorporated herein by reference). In particular, antibodies to calcyclin, calpactin I light chain, astrocytic phosphoprotein PEA-15 and tubulin-specific chaperone A are contemplated.

In accordance with the present invention, immunodetection methods are provided. Some immunodetection methods include enzyme linked immunosorbent assay (ELISA), radioimmunoassay (RIA), immunoradiometric assay, fluoroimmunoassay, chemilluminescent assay, bioluminescent assay, and Western blot to mention a few. The steps of various useful immunodetection methods have been described in the scientific literature, such as, e.g., Doolittle & Ben-Zeev, 1999; Gulbis & Galand, 1993; De Jager et al., 1993; and Nakamura et al., 1987, each incorporated herein by reference.

In general, the immunobinding methods include obtaining a sample suspected of containing a relevant polypeptide, and contacting the sample with a first antibody under conditions effective to allow the formation of immunocomplexes. In terms of antigen detection, the biological sample analyzed may be any sample that is suspected of containing an antigen, such as, for example, a tissue section or specimen, a homogenized tissue extract, a cell, or even a biological fluid.

Contacting the chosen biological sample with the antibody under effective conditions and for a period of time sufficient to allow the formation of immune complexes (primary immune complexes) is generally a matter of simply adding the antibody composition to the sample and incubating the mixture for a period of time long enough for the antibodies to form immune complexes with, i.e., to bind to, any antigens present. After this time, the sample-antibody composition, such as a tissue section, ELISA plate, dot blot or western blot, will generally be washed to remove any non-specifically bound antibody species, allowing only those antibodies specifically bound within the primary immune complexes to be detected.

In general, the detection of immunocomplex formation is well known in the art and may be achieved through the application of numerous approaches. These methods are generally based upon the detection of a label or marker, such as any of those radioactive, fluorescent, biological and enzymatic tags. Patents concerning the use of such labels include U.S. Pat. Nos. 3,817,837; 3,850,752; 3,939,350; 3,996,345; 4,277,437; 4,275,149 and 4,366,241, each incorporated herein by reference. Of course, one may find additional advantages through the use of a secondary binding ligand such as a second antibody and/or a biotin/avidin ligand binding arrangement, as is known in the art.

The antibody employed in the detection may itself be linked to a detectable label, wherein one would then simply detect this label, thereby allowing the amount of the primary immune complexes in the composition to be determined. Alternatively, the first antibody that becomes bound within the primary immune complexes may be detected by means of a second binding ligand that has binding affinity for the antibody. In these cases, the second binding ligand may be linked to a detectable label. The second binding ligand is itself often an antibody, which may thus be termed a “secondary” antibody. The primary immune complexes are contacted with the labeled, secondary binding ligand, or antibody, under effective conditions and for a period of time sufficient to allow the formation of secondary immune complexes. The secondary immune complexes are then generally washed to remove any non-specifically bound labeled secondary antibodies or ligands, and the remaining label in the secondary immune complexes is then detected.

Further methods include the detection of primary immune complexes by a two step approach. A second binding ligand, such as an antibody, that has binding affinity for the antibody is used to form secondary immune complexes, as described above. After washing, the secondary immune complexes are contacted with a third binding ligand or antibody that has binding affinity for the second antibody, again under effective conditions and for a period of time sufficient to allow the formation of immune complexes (tertiary immune complexes). The third ligand or antibody is linked to a detectable label, allowing detection of the tertiary immune complexes thus formed. This system may provide for signal amplification if this is desired.

One method of immunodetection designed by Charles Cantor uses two different antibodies. A first step biotinylated, monoclonal or polyclonal antibody is used to detect the target antigen(s), and a second step antibody is then used to detect the biotin attached to the complexed biotin. In that method the sample to be tested is first incubated in a solution containing the first step antibody. If the target antigen is present, some of the antibody binds to the antigen to form a biotinylated antibody/antigen complex. The antibody/antigen complex is then amplified by incubation in successive solutions of streptavidin (or avidin), biotinylated DNA, and/or complementary biotinylated DNA, with each step adding additional biotin sites to the antibody/antigen complex. The amplification steps are repeated until a suitable level of amplification is achieved, at which point the sample is incubated in a solution containing the second step antibody against biotin. This second step antibody is labeled, as for example with an enzyme that can be used to detect the presence of the antibody/antigen complex by histoenzymology using a chromogen substrate. With suitable amplification, a conjugate can be produced which is macroscopically visible.

Another known method of immunodetection takes advantage of the immuno-PCR (Polymerase Chain Reaction) methodology. The PCR method is similar to the Cantor method up to the incubation with biotinylated DNA, however, instead of using multiple rounds of streptavidin and biotinylated DNA incubation, the DNA/biotin/streptavidin/antibody complex is washed out with a low pH or high salt buffer that releases the antibody. The resulting wash solution is then used to carry out a PCR reaction with suitable primers with appropriate controls. At least in theory, the enormous amplification capability and specificity of PCR can be utilized to detect a single antigen molecule.

As detailed above, immunoassays are in essence binding assays. Certain immunoassays are the various types of enzyme linked immunosorbent assays (ELISAs) and radioimmunoassays (RIA) known in the art. However, it will be readily appreciated that detection is not limited to such techniques, and Western blotting, dot blotting, FACS analyses, and the like may also be used.

In one exemplary ELISA, the antibodies of the invention are immobilized onto a selected surface exhibiting protein affinity, such as a well in a polystyrene microtiter plate. Then, a test composition suspected of containing the antigen, such as a clinical sample, is added to the wells. After binding and washing to remove non-specifically bound immune complexes, the bound antigen may be detected. Detection is generally achieved by the addition of another antibody that is linked to a detectable label. This type of ELISA is a simple “sandwich ELISA”. Detection may also be achieved by the addition of a second antibody, followed by the addition of a third antibody that has binding affinity for the second antibody, with the third antibody being linked to a detectable label.

In another exemplary ELISA, the samples suspected of containing the antigen are immobilized onto the well surface and then contacted with the anti-ORF message and anti-ORF translated product antibodies of the invention. After binding and washing to remove non-specifically bound immune complexes, the bound anti-ORF message and anti-ORF translated product antibodies are detected. Where the initial anti-ORF message and anti-ORF translated product antibodies are linked to a detectable label, the immune complexes may be detected directly. Again, the immune complexes may be detected using a second antibody that has binding affinity for the first anti-ORF message and anti-ORF translated product antibody, with the second antibody being linked to a detectable label.

Another ELISA in which the antigens are immobilized involves the use of antibody competition in the detection. In this ELISA, labeled antibodies against an antigen are added to the wells, allowed to bind, and detected by means of their label. The amount of an antigen in an unknown sample is then determined by mixing the sample with the labeled antibodies against the antigen during incubation with coated wells. The presence of an antigen in the sample acts to reduce the amount of antibody against the antigen available for binding to the well and thus reduces the ultimate signal. This is also appropriate for detecting antibodies against an antigen in an unknown sample, where the unlabeled antibodies bind to the antigen-coated wells and also reduces the amount of antigen available to bind the labeled antibodies.

“Under conditions effective to allow immune complex (antigen/antibody) formation” means that the conditions preferably include diluting the antigens and/or antibodies with solutions such as BSA, bovine gamma globulin (BGG) or phosphate buffered saline (PBS)/Tween. These added agents also tend to assist in the reduction of nonspecific background. The “suitable” conditions also mean that the incubation is at a temperature or for a period of time sufficient to allow effective binding. Incubation steps are typically from about 1 to 2 to 4 hours or so, at temperatures preferably on the order of 25° C. to 27° C., or may be overnight at about 4° C. or so.

The antibodies of the present invention may also be used in conjunction with both fresh-frozen and/or formalin-fixed, paraffin-embedded tissue blocks prepared for study by immunohistochemistry (IHC). The method of preparing tissue blocks from these particulate specimens has been successfully used in previous IHC studies of various prognostic factors, and/or is well known to those of skill in the art (Brown et al., 1990; Abbondanzo et al., 1999; Allred et al., 1990).

Also contemplated in the present invention is the use of immunohistochemistry. This approach uses antibodies to detect and quantify antigens in intact tissue samples. Generally, frozen-sections are prepared by rehydrating frozen “pulverized” tissue at room temperature in phosphate buffered saline (PBS) in small plastic capsules; pelleting the particles by centrifugation; resuspending them in a viscous embedding medium (OCT); inverting the capsule and pelleting again by centrifugation; snap-freezing in −70° C. isopentane; cutting the plastic capsule and removing the frozen cylinder of tissue; securing the tissue cylinder on a cryostat microtome chuck; and cutting 25-50 serial sections.

Permanent-sections may be prepared by a similar method involving rehydration of the 50 mg sample in a plastic microfuge tube; pelleting; resuspending in 10% formalin for 4 hours fixation; washing/pelleting; resuspending in warm 2.5% agar; pelleting; cooling in ice water to harden the agar; removing the tissue/agar block from the tube; infiltrating and/or embedding the block in paraffin; and cutting up to 50 serial permanent sections.

B. Protein-Based Detection—Mass Spectrometry

By exploiting the intrinsic properties of mass and charge, mass spectrometry (MS) can resolved and confidently identified a wide variety of complex compounds, including proteins. Traditional quantitative MS has used electrospray ionization (ESI) followed by tandem MS (MS/MS) (Chen et al., 2001; Zhong et al., 2001; Wu et al., 2000) while newer quantitative methods are being developed using matrix assisted laser desorption/ionization (MALDI) followed by time of flight (TOF) MS (Bucknall et al., 2002; Mirgorodskaya et al., 2000; Gobom et al., 2000). In accordance with the present invention, one can generate mass spectrometry profiles that are useful for grading gliomas and predicting glioma patient survival, without regard for the identity of specific proteins. Alternatively, given the established links with calcyclin, calpactin I light chain, astrocytic phosphoprotein PEA-15 and tubulin-specific chaperone A, mass spectrometry may be used to look for the levels of these proteins particularly.

ESI.

ESI is a convenient ionization technique developed by Fenn and colleagues (Fenn et al., 1989) that is used to produce gaseous ions from highly polar, mostly nonvolatile biomolecules, including lipids. The sample is injected as a liquid at low flow rates (1-10 μL/min) through a capillary tube to which a strong electric field is applied. The field generates additional charges to the liquid at the end of the capillary and produces a fine spray of highly charged droplets that are electrostatically attracted to the mass spectrometer inlet. The evaporation of the solvent from the surface of a droplet as it travels through the desolvation chamber increases its charge density substantially. When this increase exceeds the Rayleigh stability limit, ions are ejected and ready for MS analysis.

A typical conventional ESI source consists of a metal capillary of typically 0.1-0.3 mm in diameter, with a tip held approximately 0.5 to 5 cm (but more usually 1 to 3 cm) away from an electrically grounded circular interface having at its center the sampling orifice, such as described by Kabarle et al. (1993). A potential difference of between 1 to 5 kV (but more typically 2 to 3 kV) is applied to the capillary by power supply to generate a high electrostatic field (10⁶ to 10⁷ V/m) at the capillary tip. A sample liquid carrying the analyte to be analyzed by the mass spectrometer is delivered to tip through an internal passage from a suitable source (such as from a chromatograph or directly from a sample solution via a liquid flow controller). By applying pressure to the sample in the capillary, the liquid leaves the capillary tip as a small highly electrically charged droplets and further undergoes desolvation and breakdown to form single or multicharged gas phase ions in the form of an ion beam. The ions are then collected by the grounded (or negatively charged) interface plate and led through an the orifice into an analyzer of the mass spectrometer. During this operation, the voltage applied to the capillary is held constant. Aspects of construction of ESI sources are described, for example, in U.S. Pat. Nos. 5,838,002; 5,788,166; 5,757,994; RE 35,413; and 5,986,258.

ESI/MS/MS.

In ESI tandem mass spectroscopy (ESI/MS/MS), one is able to simultaneously analyze both precursor ions and product ions, thereby monitoring a single precursor product reaction and producing (through selective reaction monitoring (SRM)) a signal only when the desired precursor ion is present. When the internal standard is a stable isotope-labeled version of the analyte, this is known as quantification by the stable isotope dilution method. This approach has been used to accurately measure pharmaceuticals (Zweigenbaum et al., 2000; Zweigenbaum et al., 1999) and bioactive peptides (Desiderio et al., 1996; Lovelace et al., 1991). Newer methods are performed on widely available MALDI-TOF instruments, which can resolve a wider mass range and have been used to quantify metabolites, peptides, and proteins. Larger molecules such as peptides can be quantified using unlabeled homologous peptides as long as their chemistry is similar to the analyte peptide (Duncan et al., 1993; Bucknall et al., 2002). Protein quantification has been achieved by quantifying tryptic peptides (Mirgorodskaya et al., 2000). Complex mixtures such as crude extracts can be analyzed, but in some instances sample clean up is required (Nelson et al., 1994; Gobom et al., 2000).

SIMS.

Secondary ion mass spectroscopy, or SIMS, is an analytical method that uses ionized particles emitted from a surface for mass spectroscopy at a sensitivity of detection of a few parts per billion. The sample surface is bombarded by primary energetic particles, such as electrons, ions (e.g., O, Cs), neutrals or even photons, forcing atomic and molecular particles to be ejected from the surface, a process called sputtering. Since some of these sputtered particles carry a charge, a mass spectrometer can be used to measure their mass and charge. Continued sputtering permits measuring of the exposed elements as material is removed. This in turn permits one to construct elemental depth profiles. Although the majority of secondary ionized particles are electrons, it is the secondary ions which are detected and analysis by the mass spectrometer in this method.

LD-MS and LDLPMS. Laser desorption mass spectroscopy (LD-MS) involves the use of a pulsed laser, which induces desorption of sample material from a sample site—effectively, this means vaporization of sample off of the sample substrate. This method is usually only used in conjunction with a mass spectrometer, and can be performed simultaneously with ionization if one uses the right laser radiation wavelength.

When coupled with Time-of-Flight (TOF) measurement, LD-MS is referred to as LDLPMS (Laser Desorption Laser Photoionization Mass Spectroscopy). The LDLPMS method of analysis gives instantaneous volatilization of the sample, and this form of sample fragmentation permits rapid analysis without any wet extraction chemistry. The LDLPMS instrumentation provides a profile of the species present while the retention time is low and the sample size is small. In LDLPMS, an impactor strip is loaded into a vacuum chamber. The pulsed laser is fired upon a certain spot of the sample site, and species present are desorbed and ionized by the laser radiation. This ionization also causes the molecules to break up into smaller fragment-ions. The positive or negative ions made are then accelerated into the flight tube, being detected at the end by a microchannel plate detector. Signal intensity, or peak height, is measured as a function of travel time. The applied voltage and charge of the particular ion determines the kinetic energy, and separation of fragments is due to different size causing different velocity. Each ion mass will thus have a different flight-time to the detector.

One can either form positive ions or negative ions for analysis. Positive ions are made from regular direct photoionization, but negative ion formation requires a higher powered laser and a secondary process to gain electrons. Most of the molecules that come off the sample site are neutrals, and thus can attract electrons based on their electron affinity. The negative ion formation process is less efficient than forming just positive ions. The sample constituents will also affect the outlook of a negative ion spectra.

Other advantages with the LDLPMS method include the possibility of constructing the system to give a quiet baseline of the spectra because one can prevent coevolved neutrals from entering the flight tube by operating the instrument in a linear mode. Also, in environmental analysis, the salts in the air and as deposits will not interfere with the laser desorption and ionization. This instrumentation also is very sensitive, known to detect trace levels in natural samples without any prior extraction preparations.

MALDI-TOF-MS.

Since its inception and commercial availability, the versatility of MALDI-TOF-MS has been demonstrated convincingly by its extensive use for qualitative analysis. For example, MALDI-TOF-MS has been employed for the characterization of synthetic polymers (Marie et al., 2000; Wu et al., 1998). peptide and protein analysis (Roepstorff et al., 2000; Nguyen et al., 1995), DNA and oligonucleotide sequencing (Miketova et al., 1997; Faulstich et al., 1997; Bentzley et al., 1996), and the characterization of recombinant proteins (Kanazawa et al., 1999; Villanueva et al., 1999). Recently, applications of MALDI-TOF-MS have been extended to include the direct analysis of biological tissues and single cell organisms with the aim of characterizing endogenous peptide and protein constituents (Li et al., 2000; Lynn et al., 1999; Stoeckli et al., 2001; Caprioli et al., 1997; Chaurand et al., 1999; Jespersen et al., 1999).

The properties that make MALDI-TOF-MS a popular qualitative tool—its ability to analyze molecules across an extensive mass range, high sensitivity, minimal sample preparation and rapid analysis times—also make it a potentially useful quantitative tool. MALDI-TOF-MS also enables non-volatile and thermally labile molecules to be analyzed with relative ease. It is therefore prudent to explore the potential of MALDI-TOF-MS for quantitative analysis in clinical settings, for toxicological screenings, as well as for environmental analysis. In addition, the application of MALDI-TOF-MS to the quantification of peptides and proteins is particularly relevant. The ability to quantify intact proteins in biological tissue and fluids presents a particular challenge in the expanding area of proteomics and investigators urgently require methods to accurately measure the absolute quantity of proteins. While there have been reports of quantitative MALDI-TOF-MS applications, there are many problems inherent to the MALDI ionization process that have restricted its widespread use (Kazmaier et al., 1998; Horak et al., 2001; Gobom et al., 2000; Desiderio et al., 2000). These limitations primarily stem from factors such as the sample/matrix heterogeneity, which are believed to contribute to the large variability in observed signal intensities for analytes, the limited dynamic range due to detector saturation, and difficulties associated with coupling MALDI-TOF-MS to on-line separation techniques such as liquid chromatography. Combined, these factors are thought to compromise the accuracy, precision, and utility with which quantitative determinations can be made.

Because of these difficulties, practical examples of quantitative applications of MALDI-TOF-MS have been limited. Most of the studies to date have focused on the quantification of low mass analytes, in particular, alkaloids or active ingredients in agricultural or food products (Wang et al., 1999; Jiang et al., 2000; Yang et al., 2000; Wittmann et al., 2001), whereas other studies have demonstrated the potential of MALDI-TOF-MS for the quantification of biologically relevant analytes such as neuropeptides, proteins, antibiotics, or various metabolites in biological tissue or fluid (Muddiman et al., 1996; Nelson et al., 1994; Duncan et al., 1993; Gobom et al., 2000; Wu et al., 1997; Mirgorodskaya et al., 2000). In earlier work it was shown that linear calibration curves could be generated by MALDI-TOF-MS provided that an appropriate internal standard was employed (Duncan et al., 1993). This standard can “correct” for both sample-to-sample and shot-to-shot variability. Stable isotope labeled internal standards (isotopomers) give the best result.

With the marked improvement in resolution available on modern commercial instruments, primarily because of delayed extraction (Bahr et al., 1997; Takach et al., 1997), the opportunity to extend quantitative work to other examples is now possible; not only of low mass analytes, but also biopolymers. Of particular interest is the prospect of absolute multi-component quantification in biological samples (e.g., proteomics applications).

The properties of the matrix material used in the MALDI method are critical. Only a select group of compounds is useful for the selective desorption of proteins and polypeptides. A review of all the matrix materials available for peptides and proteins shows that there are certain characteristics the compounds must share to be analytically useful. Despite its importance, very little is known about what makes a matrix material “successful” for MALDI. The few materials that do work well are used heavily by all MALDI practitioners and new molecules are constantly being evaluated as potential matrix candidates. With a few exceptions, most of the matrix materials used are solid organic acids. Liquid matrices have also been investigated, but are not used routinely.

C. Nucleic Acid Detection

In alternative embodiments for detecting protein expression, one may assay for gene transcription. For example, an indirect method for detecting protein expression is to detect mRNA transcripts from which the proteins are made. The following is a discussion of such methods, which are applicable particularly to calcyclin, calpactin I light chain, astrocytic phosphoprotein PEA-15 and tubulin-specific chaperone A in the context of the present invention.

Hybridization. There are a variety of ways by which one can assess gene expression. These methods either look at protein or at mRNA levels. Methods looking at mRNAs all fundamentally rely, at a basic level, on nucleic acid hybridization. Hybridization is defined as the ability of a nucleic acid to selectively form duplex molecules with complementary stretches of DNAs and/or RNAs. Depending on the application envisioned, one would employ varying conditions of hybridization to achieve varying degrees of selectivity of the probe or primers for the target sequence.

Typically, a probe or primer of between 13 and 100 nucleotides, preferably between 17 and 100 nucleotides in length up to 1-2 kilobases or more in length will allow the formation of a duplex molecule that is both stable and selective. Molecules having complementary sequences over contiguous stretches greater than 20 bases in length are generally preferred, to increase stability and selectivity of the hybrid molecules obtained. One will generally prefer to design nucleic acid molecules for hybridization having one or more complementary sequences of 20 to 30 nucleotides, or even longer where desired. Such fragments may be readily prepared, for example, by directly synthesizing the fragment by chemical means or by introducing selected sequences into recombinant vectors for recombinant production.

For applications requiring high selectivity, one will typically desire to employ relatively high stringency conditions to form the hybrids. For example, relatively low salt and/or high temperature conditions, such as provided by about 0.02 M to about 0.10 M NaCl at temperatures of about 50° C. to about 70° C. Such high stringency conditions tolerate little, if any, mismatch between the probe or primers and the template or target strand and would be particularly suitable for isolating specific genes or for detecting specific mRNA transcripts. It is generally appreciated that conditions can be rendered more stringent by the addition of increasing amounts of formamide.

For certain applications, for example, lower stringency conditions may be used. Under these conditions, hybridization may occur even though the sequences of the hybridizing strands are not perfectly complementary, but are mismatched at one or more positions. Conditions may be rendered less stringent by increasing salt concentration and/or decreasing temperature. For example, a medium stringency condition could be provided by about 0.1 to 0.25 M NaCl at temperatures of about 37° C. to about 55° C., while a low stringency condition could be provided by about 0.15 M to about 0.9 M salt, at temperatures ranging from about 20° C. to about 55° C. Hybridization conditions can be readily manipulated depending on the desired results.

In other embodiments, hybridization may be achieved under conditions of, for example, 50 mM Tris-HCl (pH 8.3), 75 mM KCl, 3 mM MgCl₂, 1.0 mM dithiothreitol, at temperatures between approximately 20° C. to about 37° C. Other hybridization conditions utilized could include approximately 10 mM Tris-HCl (pH 8.3), 50 mM KCl, 1.5 mM MgCl₂, at temperatures ranging from approximately 40° C. to about 72° C.

In certain embodiments, it will be advantageous to employ nucleic acids of defined sequences of the present invention in combination with an appropriate means, such as a label, for determining hybridization. A wide variety of appropriate indicator means are known in the art, including fluorescent, radioactive, enzymatic or other ligands, such as avidin/biotin, which are capable of being detected. In preferred embodiments, one may desire to employ a fluorescent label or an enzyme tag such as urease, alkaline phosphatase or peroxidase, instead of radioactive or other environmentally undesirable reagents. In the case of enzyme tags, colorimetric indicator substrates are known that can be employed to provide a detection means that is visibly or spectrophotometrically detectable, to identify specific hybridization with complementary nucleic acid containing samples.

In general, it is envisioned that the probes or primers described herein will be useful as reagents in solution hybridization, as in PCR™, for detection of expression of corresponding genes, as well as in embodiments employing a solid phase. In embodiments involving a solid phase, the test DNA (or RNA) is adsorbed or otherwise affixed to a selected matrix or surface. This fixed, single-stranded nucleic acid is then subjected to hybridization with selected probes under desired conditions. The conditions selected will depend on the particular circumstances (depending, for example, on the G+C content, type of target nucleic acid, source of nucleic acid, size of hybridization probe, etc.). Optimization of hybridization conditions for the particular application of interest is well known to those of skill in the art. After washing of the hybridized molecules to remove non-specifically bound probe molecules, hybridization is detected, and/or quantified, by determining the amount of bound label. Representative solid phase hybridization methods are disclosed in U.S. Pat. Nos. 5,843,663, 5,900,481 and 5,919,626. Other methods of hybridization that may be used in the practice of the present invention are disclosed in U.S. Pat. Nos. 5,849,481, 5,849,486 and 5,851,772. The relevant portions of these and other references identified in this section of the Specification are incorporated herein by reference.

Amplification of Nucleic Acids. Since many mRNAs are present in relatively low abundance, nucleic acid amplification greatly enhances the ability to assess expression. The general concept is that nucleic acids can be amplified using paired primers flanking the region of interest. The term “primer,” as used herein, is meant to encompass any nucleic acid that is capable of priming the synthesis of a nascent nucleic acid in a template-dependent process. Typically, primers are oligonucleotides from ten to twenty and/or thirty base pairs in length, but longer sequences can be employed. Primers may be provided in double-stranded and/or single-stranded form, although the single-stranded form is preferred.

Pairs of primers designed to selectively hybridize to nucleic acids corresponding to selected genes are contacted with the template nucleic acid under conditions that permit selective hybridization. Depending upon the desired application, high stringency hybridization conditions may be selected that will only allow hybridization to sequences that are completely complementary to the primers. In other embodiments, hybridization may occur under reduced stringency to allow for amplification of nucleic acids contain one or more mismatches with the primer sequences. Once hybridized, the template-primer complex is contacted with one or more enzymes that facilitate template-dependent nucleic acid synthesis. Multiple rounds of amplification, also referred to as “cycles,” are conducted until a sufficient amount of amplification product is produced.

The amplification product may be detected or quantified. In certain applications, the detection may be performed by visual means. Alternatively, the detection may involve indirect identification of the product via chemilluminescence, radioactive scintigraphy of incorporated radiolabel or fluorescent label or even via a system using electrical and/or thermal impulse signals.

A number of template dependent processes are available to amplify the oligonucleotide sequences present in a given template sample. One of the best known amplification methods is the polymerase chain reaction (referred to as PCR™) which is described in detail in U.S. Pat. Nos. 4,683,195, 4,683,202 and 4,800,159, and in Innis et al., 1988, each of which is incorporated herein by reference in their entirety.

A reverse transcriptase PCR™ amplification procedure may be performed to quantify the amount of mRNA amplified. Methods of reverse transcribing RNA into cDNA are well known (see Sambrook et al., 1989). Alternative methods for reverse transcription utilize thermostable DNA polymerases. These methods are described in WO 90/07641. Polymerase chain reaction methodologies are well known in the art. Representative methods of RT-PCR are described in U.S. Pat. No. 5,882,864.

Whereas standard PCR usually uses one pair of primers to amplify a specific sequence, multiplex-PCR (MPCR) uses multiple pairs of primers to amplify many sequences simultaneously (Chamberlan et al., 1990). The presence of many PCR primers in a single tube could cause many problems, such as the increased formation of misprimed PCR products and “primer dimers”, the amplification discrimination of longer DNA fragment and so on. Normally, MPCR buffers contain a Taq Polymerase additive, which decreases the competition among amplicons and the amplification discrimination of longer DNA fragment during MPCR. MPCR products can further be hybridized with gene-specific probe for verification. Theoretically, one should be able to use as many as primers as necessary. However, due to side effects (primer dimers, misprimed PCR products, etc.) caused during MPCR, there is a limit (less than 20) to the number of primers that can be used in a MPCR reaction. See also European Application No. 0 364 255 and Mueller and Wold (1989).

Another method for amplification is ligase chain reaction (“LCR”), disclosed in European Application No. 320 308, incorporated herein by reference in its entirety. U.S. Pat. No. 4,883,750 describes a method similar to LCR for binding probe pairs to a target sequence. A method based on PCR™ and oligonucleotide ligase assay (OLA), disclosed in U.S. Pat. No. 5,912,148, may also be used.

Alternative methods for amplification of target nucleic acid sequences that may be used in the practice of the present invention are disclosed in U.S. Pat. Nos. 5,843,650, 5,846,709, 5,846,783, 5,849,546, 5,849,497, 5,849,547, 5,858,652, 5,866,366, 5,916,776, 5,922,574, 5,928,905, 5,928,906, 5,932,451, 5,935,825, 5,939,291 and 5,942,391, GB Application No. 2 202 328, and in PCT Application No. PCT/US89/01025, each of which is incorporated herein by reference in its entirety.

Qbeta Replicase, described in PCT Application No. PCT/US87/00880, may also be used as an amplification method in the present invention. In this method, a replicative sequence of RNA that has a region complementary to that of a target is added to a sample in the presence of an RNA polymerase. The polymerase will copy the replicative sequence which may then be detected.

An isothermal amplification method, in which restriction endonucleases and ligases are used to achieve the amplification of target molecules that contain nucleotide 5′-[alpha-thio]-triphosphates in one strand of a restriction site may also be useful in the amplification of nucleic acids in the present invention (Walker et al., 1992). Strand Displacement Amplification (SDA), disclosed in U.S. Pat. No. 5,916,779, is another method of carrying out isothermal amplification of nucleic acids which involves multiple rounds of strand displacement and synthesis, i.e., nick translation.

Other nucleic acid amplification procedures include transcription-based amplification systems (TAS), including nucleic acid sequence based amplification (NASBA) and 3SR (Kwoh et al., 1989; Gingeras et al., PCT Application WO 88/10315, incorporated herein by reference in their entirety). European Application No. 329 822 disclose a nucleic acid amplification process involving cyclically synthesizing single-stranded RNA (“ssRNA”), ssDNA, and double-stranded DNA (dsDNA), which may be used in accordance with the present invention.

PCT Application WO 89/06700 (incorporated herein by reference in its entirety) disclose a nucleic acid sequence amplification scheme based on the hybridization of a promoter region/primer sequence to a target single-stranded DNA (“ssDNA”) followed by transcription of many RNA copies of the sequence. This scheme is not cyclic, i.e., new templates are not produced from the resultant RNA transcripts. Other amplification methods include “race” and “one-sided PCR” (Frohman, 1990; Ohara et al., 1989).

Detection of Nucleic Acids. Following any amplification, it may be desirable to separate the amplification product from the template and/or the excess primer. In one embodiment, amplification products are separated by agarose, agarose-acrylamide or polyacrylamide gel electrophoresis using standard methods (Sambrook et al., 1989). Separated amplification products may be cut out and eluted from the gel for further manipulation. Using low melting point agarose gels, the separated band may be removed by heating the gel, followed by extraction of the nucleic acid.

Separation of nucleic acids may also be effected by chromatographic techniques known in art. There are many kinds of chromatography which may be used in the practice of the present invention, including adsorption, partition, ion-exchange, hydroxylapatite, molecular sieve, reverse-phase, column, paper, thin-layer, and gas chromatography as well as HPLC.

In certain embodiments, the amplification products are visualized. A typical visualization method involves staining of a gel with ethidium bromide and visualization of bands under UV light. Alternatively, if the amplification products are integrally labeled with radio- or fluorometrically-labeled nucleotides, the separated amplification products can be exposed to x-ray film or visualized under the appropriate excitatory spectra.

In one embodiment, following separation of amplification products, a labeled nucleic acid probe is brought into contact with the amplified marker sequence. The probe preferably is conjugated to a chromophore but may be radiolabeled. In another embodiment, the probe is conjugated to a binding partner, such as an antibody or biotin, or another binding partner carrying a detectable moiety.

In particular embodiments, detection is by Southern blotting and hybridization with a labeled probe. The techniques involved in Southern blotting are well known to those of skill in the art (see Sambrook et al., 1989). One example of the foregoing is described in U.S. Pat. No. 5,279,721, incorporated by reference herein, which discloses an apparatus and method for the automated electrophoresis and transfer of nucleic acids. The apparatus permits electrophoresis and blotting without external manipulation of the gel and is ideally suited to carrying out methods according to the present invention.

Other methods of nucleic acid detection that may be used in the practice of the instant invention are disclosed in U.S. Pat. Nos. 5,840,873, 5,843,640, 5,843,651, 5,846,708, 5,846,717, 5,846,726, 5,846,729, 5,849,487, 5,853,990, 5,853,992, 5,853,993, 5,856,092, 5,861,244, 5,863,732, 5,863,753, 5,866,331, 5,905,024, 5,910,407, 5,912,124, 5,912,145, 5,919,630, 5,925,517, 5,928,862, 5,928,869, 5,929,227, 5,932,413 and 5,935,791, each of which is incorporated herein by reference.

Nucleic Acid Arrays. Microarrays comprise a plurality of polymeric molecules spatially distributed over, and stably associated with, the surface of a substantially planar substrate, e.g., biochips. Microarrays of polynucleotides have been developed and find use in a variety of applications, such as screening and DNA sequencing. One area in particular in which microarrays find use is in gene expression analysis.

In gene expression analysis with microarrays, an array of “probe” oligonucleotides is contacted with a nucleic acid sample of interest, i.e., target, such as polyA mRNA from a particular tissue type. Contact is carried out under hybridization conditions and unbound nucleic acid is then removed. The resultant pattern of hybridized nucleic acid provides information regarding the genetic profile of the sample tested. Methodologies of gene expression analysis on microarrays are capable of providing both qualitative and quantitative information.

A variety of different arrays which may be used are known in the art. The probe molecules of the arrays which are capable of sequence specific hybridization with target nucleic acid may be polynucleotides or hybridizing analogues or mimetics thereof, including: nucleic acids in which the phosphodiester linkage has been replaced with a substitute linkage, such as phosphorothioate, methylimino, methylphosphonate, phosphoramidate, guanidine and the like; nucleic acids in which the ribose subunit has been substituted, e.g., hexose phosphodiester; peptide nucleic acids; and the like. The length of the probes will generally range from 10 to 1000 nts, where in some embodiments the probes will be oligonucleotides and usually range from 15 to 150 nts and more usually from 15 to 100 nts in length, and in other embodiments the probes will be longer, usually ranging in length from 150 to 1000 nts, where the polynucleotide probes may be single- or double-stranded, usually single-stranded, and may be PCR fragments amplified from cDNA.

The probe molecules on the surface of the substrates will correspond to selected genes being analyzed and be positioned on the array at a known location so that positive hybridization events may be correlated to expression of a particular gene in the physiological source from which the target nucleic acid sample is derived. The substrates with which the probe molecules are stably associated may be fabricated from a variety of materials, including plastics, ceramics, metals, gels, membranes, glasses, and the like. The arrays may be produced according to any convenient methodology, such as preforming the probes and then stably associating them with the surface of the support or growing the probes directly on the support. A number of different array configurations and methods for their production are known to those of skill in the art and disclosed in U.S. Pat. Nos. 5,445,934, 5,532,128, 5,556,752, 5,242,974, 5,384,261, 5,405,783, 5,412,087, 5,424,186, 5,429,807, 5,436,327, 5,472,672, 5,527,681, 5,529,756, 5,545,531, 5,554,501, 5,561,071, 5,571,639, 5,593,839, 5,599,695, 5,624,711, 5,658,734, 5,700,637, and 6,004,755.

Following hybridization, where non-hybridized labeled nucleic acid is capable of emitting a signal during the detection step, a washing step is employed where unhybridized labeled nucleic acid is removed from the support surface, generating a pattern of hybridized nucleic acid on the substrate surface. A variety of wash solutions and protocols for their use are known to those of skill in the art and may be used.

Where the label on the target nucleic acid is not directly detectable, one then contacts the array, now comprising bound target, with the other member(s) of the signal producing system that is being employed. For example, where the label on the target is biotin, one then contacts the array with streptavidin-fluorescer conjugate under conditions sufficient for binding between the specific binding member pairs to occur. Following contact, any unbound members of the signal producing system will then be removed, e.g., by washing. The specific wash conditions employed will necessarily depend on the specific nature of the signal producing system that is employed, and will be known to those of skill in the art familiar with the particular signal producing system employed.

The resultant hybridization pattern(s) of labeled nucleic acids may be visualized or detected in a variety of ways, with the particular manner of detection being chosen based on the particular label of the nucleic acid, where representative detection means include scintillation counting, autoradiography, fluorescence measurement, calorimetric measurement, light emission measurement and the like.

Prior to detection or visualization, where one desires to reduce the potential for a mismatch hybridization event to generate a false positive signal on the pattern, the array of hybridized target/probe complexes may be treated with an endonuclease under conditions sufficient such that the endonuclease degrades single stranded, but not double stranded DNA. A variety of different endonucleases are known and may be used, where such nucleases include: mung bean nuclease, S1 nuclease, and the like. Where such treatment is employed in an assay in which the target nucleic acids are not labeled with a directly detectable label, e.g., in an assay with biotinylated target nucleic acids, the endonuclease treatment will generally be performed prior to contact of the array with the other member(s) of the signal producing system, e.g., fluorescent-streptavidin conjugate. Endonuclease treatment, as described above, ensures that only end-labeled target/probe complexes having a substantially complete hybridization at the 3′ end of the probe are detected in the hybridization pattern.

Following hybridization and any washing step(s) and/or subsequent treatments, as described above, the resultant hybridization pattern is detected. In detecting or visualizing the hybridization pattern, the intensity or signal value of the label will be not only be detected but quantified, by which is meant that the signal from each spot of the hybridization will be measured and compared to a unit value corresponding the signal emitted by known number of end-labeled target nucleic acids to obtain a count or absolute value of the copy number of each end-labeled target that is hybridized to a particular spot on the array in the hybridization pattern.

D. Enzyme Activity

In some cases, the presence of an IDH1 or IDH2 mutation can be detected by measuring enzyme activity. For example, IDH1 and IDH2 enzymes can be assessed for oxidative decarboxylation of isocitrate to αKG with NADP+ as cofactor. For example, the presence of an IDH1 mutation can be detected by measuring 2-HG production.

E. Combination Diagnosis

In one aspect of this document, the presence or absence of an IDH1 or IDH2 mutation can be used in combination with current testing methods to determine a diagnosis of CCA. For example, testing for the presence of malignant cells by cytopathology, histopathology or fluorescence in situ hybridization can be used in combination with screening for IDH1 or IDH2 mutations to aid in the diagnosis of CCA. In one aspect of this document, the presence or absence of an IDH1 or IDH2 mutation can be used in combination with current testing methods to determine a prognosis of a mammal with CCA. For example, the presence of malignant cells by cytopathology, histopathology or fluorescence in situ hybridization can be used in combination with screening for IDH1 or IDH2 mutations to determine the prognosis of a mammal diagnosed with CCA.

2-hydroxyglutarate (2-HG) is suspected as playing a significant role in CCA. According to Borger & Zhu (2012), the prevailing view is that 2-HG acts as an “oncometabolite.” The structural similarity between 2-HG and α-ketoglutarate (αKG) has been shown to disrupt normal αKG function. 2-HG is the result of aberrant activity of mutated IDH enzymes, and 2-HG levels have been shown to be elevated in tumors (e.g., gliomas) exhibited IDH mutations. 2-HG can be detected using both NMR and mass spectroscopy.

### 5. THERAPIES

There are three general approaches to the treatment of CCA. First, there are standard therapies currently in use. Second, there are targeted therapies directed at the mutant IDH enzymes. And third, there are targeted therapies that attempt to inhibit the effect of 2-HG produced by the aberrant activity of mutated IDH.

A. Current Standard of Care

Cholangiocarcinoma is considered to be an incurable and rapidly lethal disease unless all the tumors can be fully resected. Since the operability of the tumor can only be assessed during surgery in most cases, a majority of patients undergo exploratory surgery unless there is already a clear indication that the tumor is inoperable. Adjuvant therapy followed by liver transplantation may have a role in treatment of certain unresectable cases.

If the tumor can be removed surgically, patients may receive adjuvant chemotherapy or radiation therapy after the operation to improve the chances of cure. If the tissue margins are negative (i.e., the tumor has been totally excised), adjuvant therapy is of uncertain benefit. Both positive and negative results have been reported with adjuvant radiation therapy in this setting, and no prospective randomized controlled trials have been conducted as of March 2007. Adjuvant chemotherapy appears to be ineffective in patients with completely resected tumors. The role of combined chemoradiotherapy in this setting is unclear. However, if the tumor tissue margins are positive, indicating that the tumor was not completely removed via surgery, then adjuvant therapy with radiation and possibly chemotherapy is generally recommended based on the available data.

The majority of cases of cholangiocarcinoma present as inoperable (unresectable) disease in which case patients are generally treated with palliative chemotherapy, with or without radiotherapy. Chemotherapy has been shown in a randomized controlled trial to improve quality of life and extend survival in patients with inoperable cholangiocarcinoma. There is no single chemotherapy regimen which is universally used, and enrollment in clinical trials is often recommended when possible. Chemotherapy agents used to treat cholangiocarcinoma include 5-fluorouracil with leucovorin, gemcitabine as a single agent, or gemcitabine plus cisplatin, irinotecan, or capecitabine. A small pilot study suggested possible benefit from the tyrosine kinase inhibitor erlotinib in patients with advanced cholangiocarcinoma.

Photodynamic therapy, an experimental approach in which patients are injected with a light-sensitizing agent and light is then applied endoscopically directly to the tumor, has shown promising results compared to supportive care in two small randomized controlled trials. However, its ultimate role in the management of cholangiocarcinoma is unclear at present. Photodynamic Therapy has been shown to improve survival and quality of life.

B. IDH Targeted Therapies

In certain embodiments, one may wish to target a mutated IDH enzyme. Inhibition can be achieved by use of a double-stranded RNA (dsRNA) directed to an mRNA for IDH. In such embodiments, the dsRNA mediates the reduction of the expression of IDH.

RNA interference (also referred to as “RNA-mediated interference” or RNAi) is a mechanism by which gene expression can be reduced or eliminated. Double-stranded RNA (dsRNA) has been observed to mediate the reduction, which is a multi-step process. dsRNA activates post-transcriptional gene expression surveillance mechanisms that appear to function to defend cells from virus infection and transposon activity (Fire et al., 1998; Grishok et al., 2000; Ketting et al., 1999; Lin and Avery et al., 1999; Montgomery et al., 1998; Sharp and Zamore, 2000; Tabara et al., 1999). Activation of these mechanisms targets mature, dsRNA-complementary mRNA for destruction. RNAi offers major experimental advantages for study of gene function. These advantages include a very high specificity, ease of movement across cell membranes, and prolonged down-regulation of the targeted gene (Fire et al., 1998; Grishok et al., 2000; Ketting et al., 1999; Lin and Avery et al., 1999; Montgomery et al., 1998; Sharp et al., 1999; Sharp and Zamore, 2000; Tabara et al., 1999). It is generally accepted that RNAi acts post-transcriptionally, targeting RNA transcripts for degradation. It appears that both nuclear and cytoplasmic RNA can be targeted (Bosher and Labouesse, 2000).

siRNAs must be designed so that they are specific and effective in suppressing the expression of the genes of interest. Methods of selecting the target sequences, i.e., those sequences present in the gene or genes of interest to which the siRNAs will guide the degradative machinery, are directed to avoiding sequences that may interfere with the siRNA's guide function while including sequences that are specific to the gene or genes. Typically, siRNA target sequences of about 21 to 23 nucleotides in length are most effective. This length reflects the lengths of digestion products resulting from the processing of much longer RNAs as described above (Montgomery et al., 1998). siRNA are well known in the art. For example, siRNA and double-stranded RNA have been described in U.S. Pat. Nos. 6,506,559 and 6,573,099, as well as in U.S. Patent Applications 2003/0051263, 2003/0055020, 2004/0265839, 2002/0168707, 2003/0159161, and 2004/0064842, all of which are herein incorporated by reference in their entirety.

Several further modifications to siRNA sequences have been suggested in order to alter their stability or improve their effectiveness. It is suggested that synthetic complementary 21-mer RNAs having di-nucleotide overhangs (i.e., 19 complementary nucleotides +3′ non-complementary dimers) may provide the greatest level of suppression. These protocols primarily use a sequence of two (2′-deoxy) thymidine nucleotides as the di-nucleotide overhangs. These dinucleotide overhangs are often written as dTdT to distinguish them from the typical nucleotides incorporated into RNA.

The literature has indicated that the use of dT overhangs is primarily motivated by the need to reduce the cost of the chemically synthesized RNAs. It is also suggested that the dTdT overhangs might be more stable than UU overhangs, though the data available shows only a slight (<20%) improvement of the dTdT overhang compared to an siRNA with a UU overhang.

Short hairpin RNA (shRNA) is a sequence of RNA that makes a tight hairpin turn that can be used to silence gene expression. shRNA is transcribed by RNA polymerase III. shRNA production in a mammalian cell can sometimes cause the cell to mount an interferon response as the cell seeks to defend itself from what it perceives as viral attack. Paddison et al. (2002) examined the importance of stem and loop length, sequence specificity, and presence of overhangs in determining shRNA activity. The authors found some interesting results. For example, they showed that the length of the stem and loop of functional shRNAs could vary. Stem lengths could range anywhere from 25 to 29 nt and loop size could range between 4 to 23 nt without affecting silencing activity. Presence of G-U mismatches between the 2 strands of the shRNA stem did not lead to a decrease in potency. Complementarity between the portion of the stem that binds to the target mRNA (antisense strand) and the mRNA, on the other hand, was shown to be critical. Single base mismatches between the antisense strand of the stem and the mRNA abolished silencing. It has been reported that presence of 2 nt 3′-overhangs is critical for siRNA activity (Elbashir et al., 2001). Presence of overhangs on shRNAs, however, did not seem to be important. Some of the functional shRNAs that were either chemically synthesized or in vitro transcribed, for example, did not have predicted 3′ overhangs.

dsRNA can be synthesized using well-described methods (Fire et al., 1998). Briefly, sense and antisense RNA are synthesized from DNA templates using T7 polymerase (MEGAscript, Ambion). After the synthesis is complete, the DNA template is digested with DNaseI and RNA purified by phenol/chloroform extraction and isopropanol precipitation. RNA size, purity and integrity are assayed on denaturing agarose gels. Sense and antisense RNA are diluted in potassium citrate buffer and annealed at 80° C. for 3 min to form dsRNA. As with the construction of DNA template libraries, a procedures may be used to aid this time intensive procedure. The sum of the individual dsRNA species is designated as a “dsRNA library.”

The making of siRNAs has been mainly through direct chemical synthesis; through processing of longer, double-stranded RNAs through exposure to Drosophila embryo lysates; or through an in vitro system derived from S2 cells. Use of cell lysates or in vitro processing may further involve the subsequent isolation of the short, 21-23 nucleotide siRNAs from the lysate, etc., making the process somewhat cumbersome and expensive. Chemical synthesis proceeds by making two single-stranded RNA-oligomers followed by the annealing of the two single-stranded oligomers into a double-stranded RNA. Methods of chemical synthesis are diverse. Non-limiting examples are provided in U.S. Pat. Nos. 5,889,136, 4,415,723, and 4,458,066, expressly incorporated herein by reference, and in Wincott et al. (1995).

WO 99/32619 and WO 01/68836 suggest that RNA for use in siRNA may be chemically or enzymatically synthesized. Both of these texts are incorporated herein in their entirety by reference. The enzymatic synthesis contemplated in these references is by a cellular RNA polymerase or a bacteriophage RNA polymerase (e.g., T3, T7, SP6) via the use and production of an expression construct as is known in the art. For example, see U.S. Pat. No. 5,795,715. The contemplated constructs provide templates that produce RNAs that contain nucleotide sequences identical to a portion of the target gene. The length of identical sequences provided by these references is at least 25 bases, and may be as many as 400 or more bases in length. An important aspect of this reference is that the authors contemplate digesting longer dsRNAs to 21-25mer lengths with the endogenous nuclease complex that converts long dsRNAs to siRNAs in vivo. They do not describe or present data for synthesizing and using in vitro transcribed 21-25mer dsRNAs. No distinction is made between the expected properties of chemical or enzymatically synthesized dsRNA in its use in RNA interference.

Similarly, WO 00/44914, incorporated herein by reference, suggests that single strands of RNA can be produced enzymatically or by partial/total organic synthesis. Preferably, single-stranded RNA is enzymatically synthesized from the PCR products of a DNA template, preferably a cloned cDNA template and the RNA product is a complete transcript of the cDNA, which may comprise hundreds of nucleotides. WO 01/36646, incorporated herein by reference, places no limitation upon the manner in which the siRNA is synthesized, providing that the RNA may be synthesized in vitro or in vivo, using manual and/or automated procedures. This reference also provides that in vitro synthesis may be chemical or enzymatic, for example using cloned RNA polymerase (e.g., T3, T7, SP6) for transcription of the endogenous DNA (or cDNA) template, or a mixture of both. Again, no distinction in the desirable properties for use in RNA interference is made between chemically or enzymatically synthesized siRNA.

U.S. Pat. No. 5,795,715 reports the simultaneous transcription of two complementary DNA sequence strands in a single reaction mixture, wherein the two transcripts are immediately hybridized. The templates used are preferably of between 40 and 100 base pairs, and which is equipped at each end with a promoter sequence. The templates are preferably attached to a solid surface. After transcription with RNA polymerase, the resulting dsRNA fragments may be used for detecting and/or assaying nucleic acid target sequences.

Several groups have developed expression vectors that continually express siRNAs in stably transfected mammalian cells (Brummelkamp et al., 2002; Lee et al., 2002; Miyagishi and Taira, 2002; Paddison et al., 2002; Paul et al., 2002; Sui et al., 2002; Yu et al., 2002), Some of these plasmids are engineered to express shRNAs lacking poly (A) tails (Brummelkamp et al., 2002; Paddison et al., 2002; Paul et al., 2002; Yu et al., 2002). Transcription of shRNAs is initiated at a polymerase III (pol III) promoter and is believed to be terminated at position 2 of a 4-5-thymine transcription termination site. shRNAs are thought to fold into a stem-loop structure with 3′ UU-overhangs. Subsequently, the ends of these shRNAs are processed, converting the shRNAs into ˜21 nt siRNA-like molecules (Brummelkamp et al., 2002). The siRNA-like molecules can, in turn, bring about gene-specific silencing in the transfected mammalian cells.

More generally, most any oligo- or polynucleotide may be made by any technique known to one of ordinary skill in the art, such as chemical synthesis, enzymatic production or biological production. Non-limiting examples of a synthetic nucleic acid (e.g., a synthetic oligonucleotide), include a nucleic acid made by in vitro chemically synthesis using phosphotriester, phosphite or phosphoramidite chemistry and solid phase techniques such as described in EP 266,032, incorporated herein by reference, or via deoxynucleoside H-phosphonate intermediates as described by Froehler et al., 1986 and U.S. Pat. No. 5,705,629, each incorporated herein by reference. In the methods of the present invention, one or more oligonucleotide may be used. Various different mechanisms of oligonucleotide synthesis have been disclosed in for example, U.S. Pat. Nos. 4,659,774, 4,816,571, 5,141,813, 5,264,566, 4,959,463, 5,428,148, 5,554,744, 5,574,146, 5,602,244, each of which is incorporated herein by reference.

A non-limiting example of an enzymatically produced nucleic acid include one produced by enzymes in amplification reactions such as PCR™ (see for example, U.S. Pat. No. 4,683,202 and U.S. Pat. No. 4,682,195, each incorporated herein by reference), or the synthesis of an oligonucleotide described in U.S. Pat. No. 5,645,897, incorporated herein by reference. A non-limiting example of a biologically produced nucleic acid includes a recombinant nucleic acid produced (i.e., replicated) in a living cell, such as a recombinant DNA vector replicated in bacteria (see for example, Sambrook et al. 2001, incorporated herein by reference).

C. 2-HG Targeted Therapies

Small molecules are currently being developed to interfere with the pathologic signaling effects of 2-HG. For example, WO 2011/072174 (incorporated by reference) discloses compounds useful in cells having α-hydroxyl neoactivity, including those have elevated quantities of 2-HG. Similarly, WO 2011/143160 (incorporated by reference) discloses glutaminase inhibitors effective in cancers having mutant IDH genes producing 2-HG. These types of inhibitors find particular utility when patients are identified as having aberrant IDH genes.

### 6. EXAMPLES

The following examples are included to demonstrate particular embodiments of the invention. It should be appreciated by those of skill in the art that the techniques disclosed in the examples which follow represent techniques discovered by the inventor to function well in the practice of the invention. However, those of skill in the art should, in light of the present disclosure, appreciate that many changes can be made in the specific embodiments which are disclosed and still obtain a like or similar result without departing from the spirit and scope of the invention.

### Example 1

### Identification of IDH1 and IDH2 Mutations in Cholangiocarcinoma

DNA Samples.

Ninety-four patient surgically resected primary CCA (67 intrahepatic and 27 extrahepatic) fresh-frozen paraffin-embedded (FFPE) tissue blocks and corresponding hematoxylin and eosin (H&E) stained slides were retrieved from Mayo Clinic tissue archives for IDH1 and IDH2 mutation analysis. Retrieved H&E slides were reviewed by a pathologist to select tissue blocks with adequate tumor for subsequent DNA testing. Eight serial 5-μm sections were then cut from selected tissue blocks; and areas comprising of >20% tumor were macrodissected. Samples were then dewaxed by xylene wash (20 minutes) followed by two 100% ethanol washes. The remainder of the DNA extraction procedure was performed using a QIAamp DNA Mini Kit (QIAGEN, Valencia, Calif.) as recommended by the manufacturer.

PCR amplification of the IDH1 region containing codon 132 was performed using primer pair 1 (fwd-5′-TCAGAGAAGCCATTATCTGCAAAAATAT-3′ (SEQ ID NO:5) and rev-5′-biotin-GGCCATGAAAAAAAAAACATGC-3′ (SEQ ID NO:6); product size 143 base pairs). The IDH2 region containing codon 170 was amplified using primer pair 2 (fwd-5′-AAAACATCCCACGCCTAGTCC-3′ (SEQ ID NO:7) and rev-5′-biotin-GTGCCCAGGTCAGTGGATC-3′ (SEQ ID NO:8); product size 110 base pairs). PCR for IDH1 and IDH2 was performed in 25 μL reaction volumes per specimen containing 2.0 μL of DNA template (25-250 μg/mL), 18.3 μL nuclease free H₂O, 2.5 μL 10×PCR buffer (Sigma), 1.0 μL 50 mM MgCl₂, 0.5 μL 10 mM dNTP mix, 0.3 μL forward primer (25 μM), 0.3 μL reverse primer (25 μM), and 0.1 μL Platinum Taq. PCR conditions were as follows: 94° C. for 120 sec, 40 cycles of 94° C. for 30 sec, 61° C. for 30 sec, and 72° C. for 60 sec, followed by 72° C. for 10 min.

Pyrosequencing was performed on a Qiagen PyroMark Q24 system according to the manufacturer's protocol. Ten μl PCR product, 2 μL Streptavidin Sepharose High Performance beads (GE Healthcare Bio-Sciences AB, Sweden Uppsala), 28 μL nanopure water, and 40 μL binding buffer (Qiagen) were mixed and agitated on a plate shaker for 10 min at 1,800 rpm to adequately bind the PCR product to the beads. The beads were then captured using the vacuum prep workstation (Qiagen), washed in 40 mL of 70% ethanol, denatured with 40 ml denaturation buffer (Qiagen), followed by washing in 50 ml washing buffer (Qiagen) per manufacturer protocol. The beads were then released in a 24 well pyrosequencing plate and purified DNA samples were annealed to the sequencing primer (IDH1-PySeq: 5′-TGGGTAAAACCTATCATC-3′ (SEQ ID NO:1) or IDH2-PySeq: 5′-AAGCCCATCACCATT-3′ (SEQ ID NO:2); 0.3 μM) in 25 μl annealing buffer (Qiagen) for 2 min at 80° C., and cooled for 5 min at room temperature. Pyrosequencing was performed on the Pyromark Q24 instrument using PyroGold Reagents (Qiagen) using the dispensation order of GATACGTAGCATGTCAT (SEQ ID NO:3) for IDH1 and CGTCATCGTACAC (SEQ ID NO:4) for IDH2 analyses. Pyrograms were manually interpreted and evaluated using the PyroMark Q24 software. Verification of mutations was performed by Sanger sequencing using primers and PCR conditions previously described (Hartmann et al., 2009), with the exception that universal primer sequencing tags were added to each of the primers.

Results.

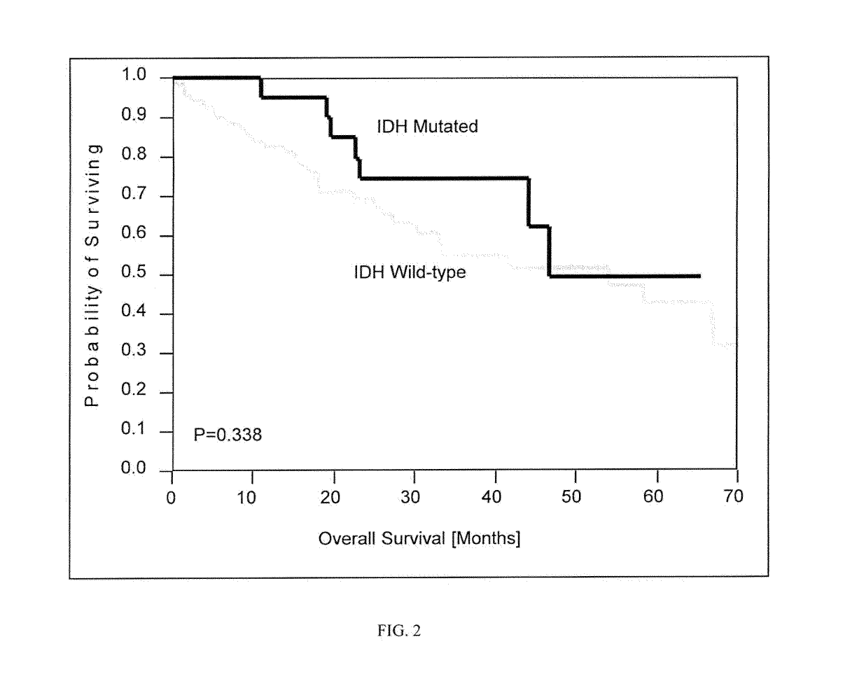

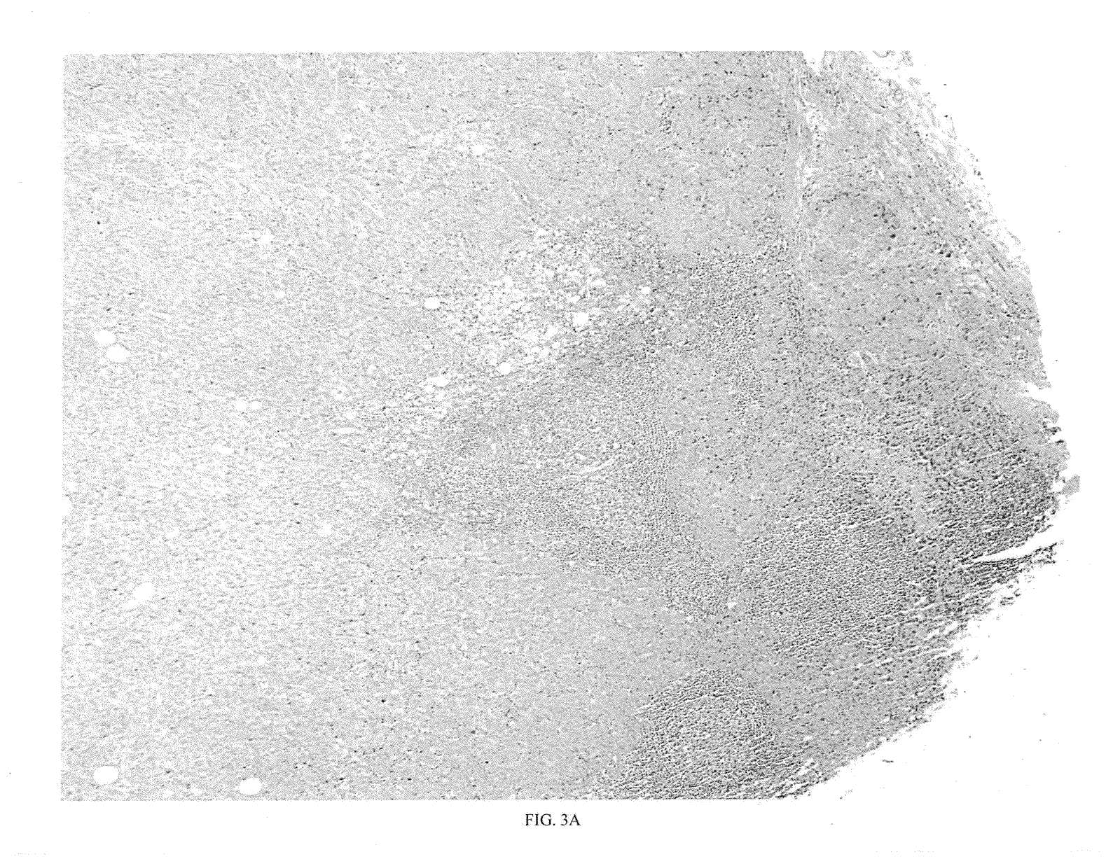

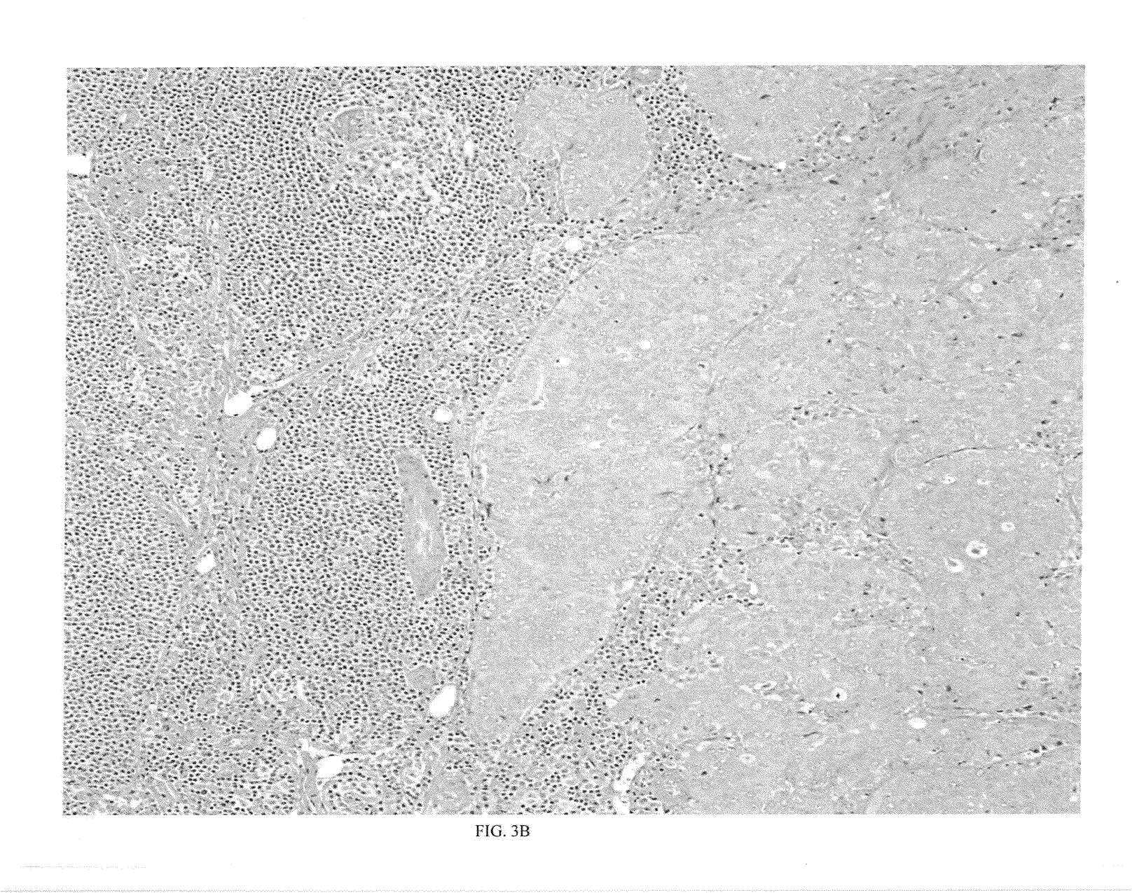

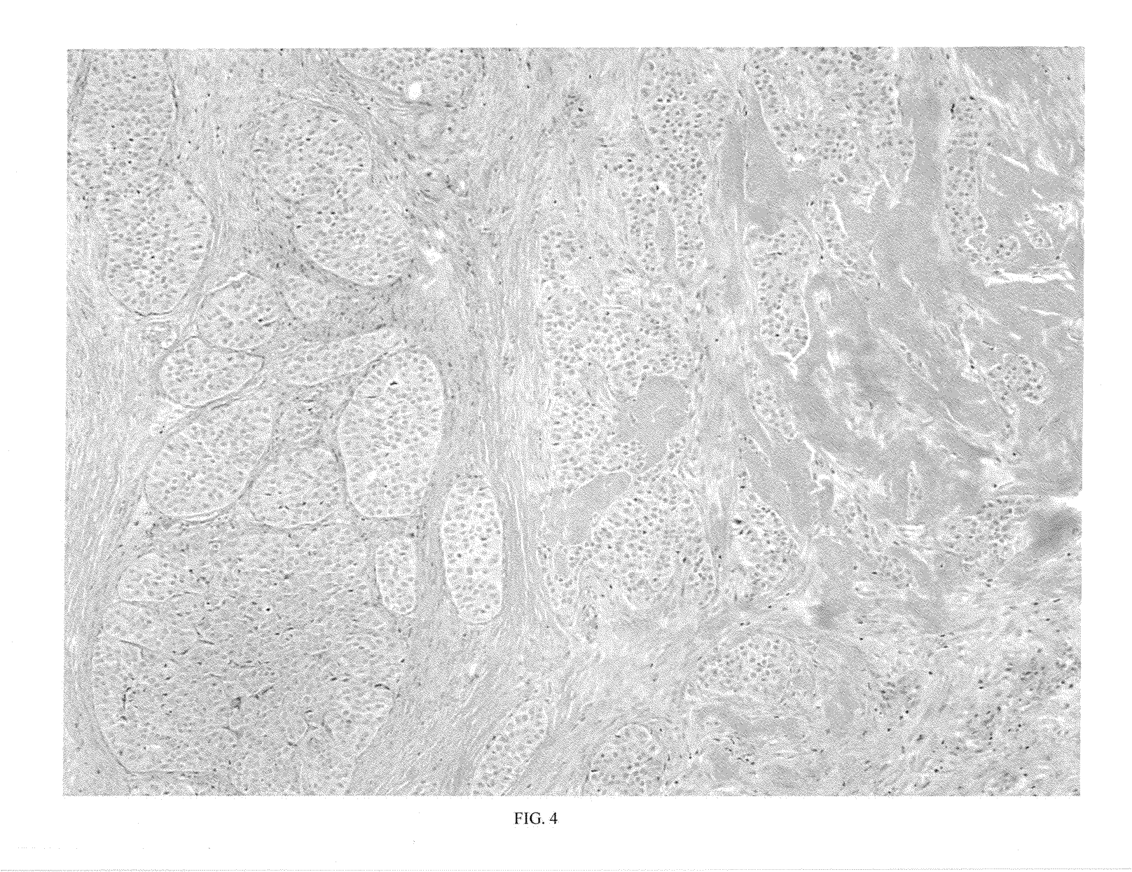

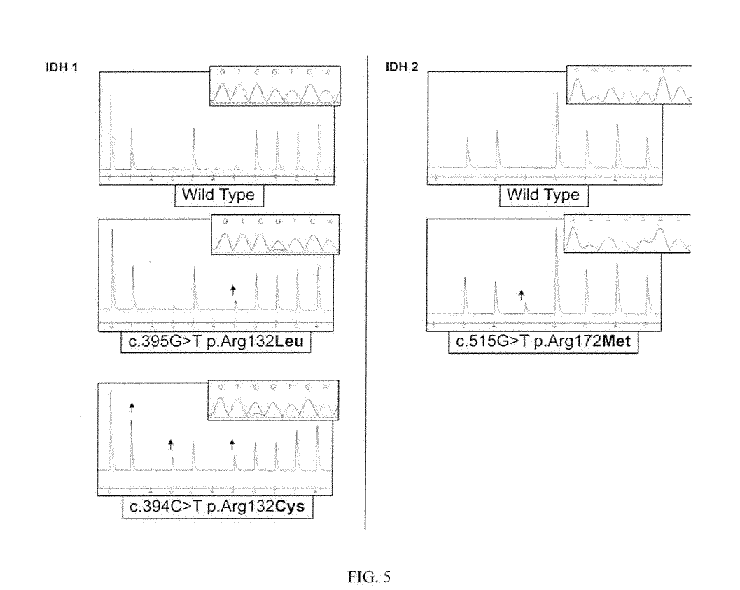

The clinicopathologic features of patients based on IDH1 and IDH2 mutation status are summarized in Table 1. IDH mutations were more frequently observed in intrahepatic CCA compared to extrahepatic CCA (28% vs. 7%, respectively; P=0.030). There were no significant differences in age, gender, lymph node metastasis, or associated PSC status when comparing patients with and without IDH mutations. Patients with an IDH1 and IDH2 gene mutation appeared to have better overall survival a year following surgical resection when compared to patients without an IDH1 or IDH2 mutation (95% vs. 83%, respectively; FIG. 2). However, patients with an IDH gene mutation had a median overall survival of 46.7 months, which was not significantly different (P=0.338; FIG. 2) than 53.9 months for patients without the gene mutation.

TABLE 1
Summary of Clinicopathologic Features by IDH Status

| | IDH Mutation Status | | |
|---|---|---|---|
| Variable | Mutation | No Mutation | P Value |
| Specimens (N = 94) | 21 | 73 | |
| Mean Age | 61.7 | 62.5 | 0.741 |
| Gender | | | |
| Male (n = 53) | 8 (15%) | 45 (85%) | 0.055 |
| Female (n = 41) | 13 (32%) | 28 (68%) | |
| Location | | | |
| Intrahepatic (n = 67) | 19 (28%) | 48 (72%) | 0.030 |
| Extrahepatic (n = 27) | 2 (7%) | 25 (93%) | |
| LN Mets | | | |
| Yes (n = 32) | 8 (25%) | 24 (75%) | 0.657 |
| No (n = 62) | 13 (21%) | 49 (79%) | |
| Primary Sclerosing Cholangitis | | | |
| Yes (n = 6) | 0 (0%) | 6 (100%) | 0.332 |
| No (n = 88) | 21 (24%) | 67 (76%) | |

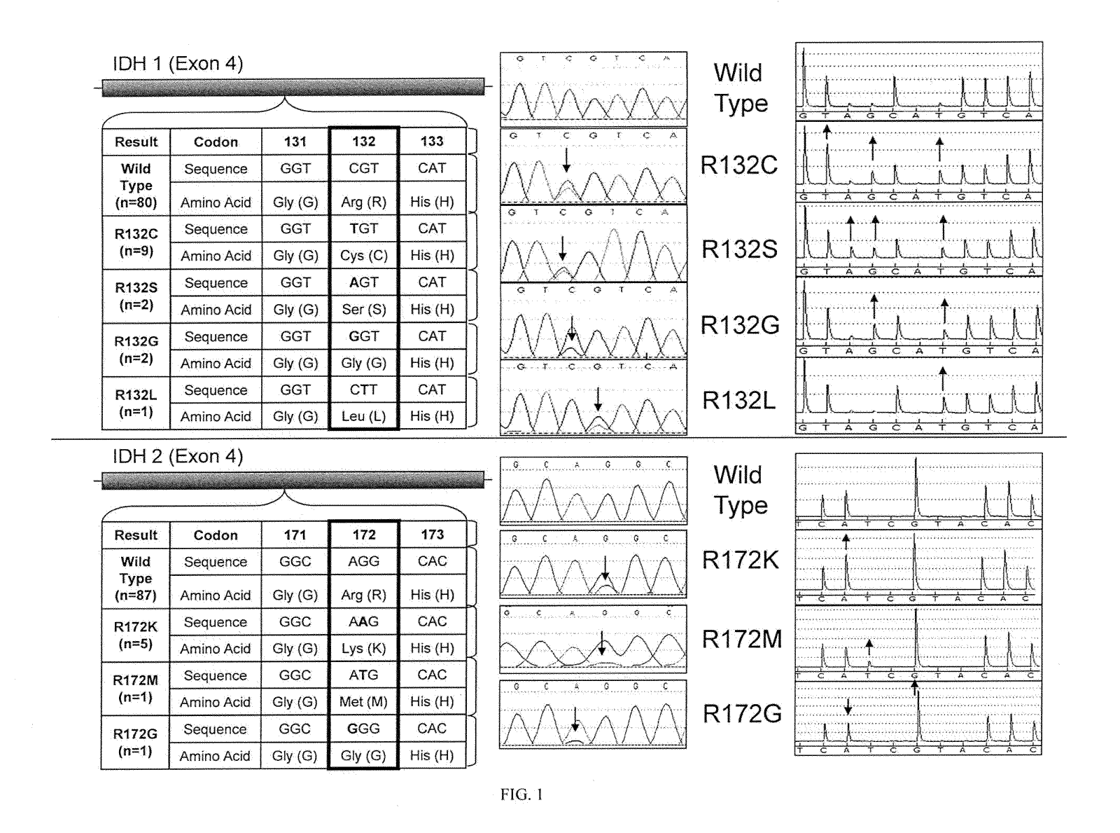

The results of this study show for the first time that IDH1 and IDH2 genes are mutated in CCA. The mutations observed in this study are similar to those observed in gliomas with IDH1 mutations being more prevalent that IDH2 mutations (67% vs. 33%%; FIG. 1). Unlike previous IDH studies, there were no R132H mutations detected in this study. However, there were 14 CCA specimens that harbored the R132c/G/S/L mutations.

IDH1 and IDH2 mutations were more frequently observed in intrahepatic CCA's compared to extrahepatic CCA (28% vs. 7%, respectively; P=0.030). The data from this study also shows that patients with an IDH1 and IDH2 mutation appear to have better overall survival immediately following surgical resection; however, these results were underpowered and not statistically significant. An important aspect of this study is that the patient population comprised exclusively of resectable CCA specimens. This suggests that IDH mutations occur early in CCA carcinogenesis which increases its potential value as a diagnostic, prognostic and/or therapeutic marker.

In conclusion, the results of this study demonstrate for the first time that both IDH1 and IDH2 mutations are present in a subset of CCA's. These findings have potential clinical implications including the prospective use of 2-HG as a biomarker for earlier detection of CCA. IDH1 and IDH2 mutations status may also increase the ability to detect CCA when therapies are more effective. Lastly, and maybe most importantly, patients with IDH mutations may be amenable to future targeted therapies that will hopefully increase survival in patients with CCA.

### Example 2

### Metastatic Cholangiocarcinomas with IDH1 and IDH2 Mutations

Methods and Materials.

Cases. Previously identified cases of primary cholangiocarcinoma with known IDH1/IDH2 mutations and IDH1/IDH2 wild-type were selected. The medical records of the patients were reviewed to determine patients who presented with or subsequently developed metastases. Formalin-fixed paraffin embedded tissue sections of the paired primary tumor and metastases were obtained in cases of biopsy-proven metastatic disease. Haematoxylin and eosin (H&E) stained sections, as well as unstained 5 micron thick sections were prepared from the tumor blocks. The H&E slides were reviewed by pathologists (RPG and LZ) to assess morphological characteristics, and to select tissue blocks with adequate tumor for subsequent DNA testing. A minimum of 20% tumor was required for DNA extraction.

DNA Extraction and Sequencing. DNA extraction and sequencing was performed using previously published and validated methods (Kipp et al., 2012). All specimens were pyrosequenced assessing for IDH1 (codon 132) and IDH2 (codon 172) mutations. Positive results were verified by Sanger sequencing.

Results.

IDH1/IDH2 Mutant. Twenty-one specimens had been previously identified as having an IDH1 (n=14) or IDH2 (n=7) mutation. Of these, 6 of 21 developed metastases and had tissue sections available for testing. This included 4 males and 2 females with an age range of 51-65 years (median 58 years). Two of the 6 patients presented with metastatic disease. The remaining 4 developed metastases within 2 years of the primary diagnosis (range=183-537 days). All 6 cases displayed the same IDH1 (n=5) or IDH2 (n=1) mutation in the paired primary and metastatic tumors. Microscopically, the tumors were moderately differentiated (n=1) and poorly differentiated (n=5). In 3 of 6 cases, the metastatic tumors were characterized by a nested morphology similar to that observed in the primary tumor.

IDH1/IDH2 wild-type. Of the previously identified 79 IDH wild-type cholangiocarcinomas, 5 with biopsy-proven metastases were selected, including 4 with metastases at presentation and 1 with a metastasis within 8 months of primary diagnosis. Each primary and metastasis was IDH1/IDH2 wild-type. The tumors and metastases were moderately differentiated.

Conclusion.

IDH mutations were initially identified in glial tumors and acute myeloid leukemia (Mardis et al., 2009; Yan, Parsons et al., 2009). Subsequently, these mutations were recognized in chondrosarcoma (Amary et al., 2011), rarely in prostate cancer (Ghiam, Cairns et al. 2011) and now recently in cholangiocarcinoma by the inventors (Kipp et al., 2012) and others (Borger et al., 2012).

The results of the current study confirm concordance of IDH mutant status between primary and metastatic cholangiocarcinomas in the 6 samples tested. Interestingly, the study illustrates that IDH mutations did not occur during tumor progression of wild-type tumors in 5 selected cases. Taken together, these data confirm that either tissue from metastatic or primary cholangiocarcinoma can be utilized for IDH mutation assessment as part of genotype-directed therapeutic studies.

With respect to colorectal carcinoma, it has been reported that metastatic tumor tissue demonstrates concordance with primary tumors in terms of KRAS and BRAF mutation status (Santini et al., 2010; Kawamoto, Tsuchihara et al., 2012). By contrast, for breast cancer, the current standard of care (Hammond et al., 2010), (Wolff et al., 2007) includes evaluation of each newly diagnosed metastasis for ER, PR and HER2 status in view of discordance between primaries and metastases in published series (Masood and Bui 2000; Hoelhagel et al., 2010) (Holdaway and Bowditch 1983).

Targeted therapies are anticipated to play an increasing role in the management of tumors. This is hoped to lead to better efficacy with decreased toxicity and as already alluded to have become part of the armamentarium in the treatment of colorectal, lung and breast carcinoma as well as melanoma recently.

This study extends on previously published report describing IDH mutations in cholangiocarcinomas (Kipp et al., 2012) with the confirmation of concordance of IDH mutations status between primary cholangiocarcinomas and metastases. Evaluation of either primary or metastatic cholangiocarcinoma tissue is acceptable for determination of IDH mutation status. These results suggest that IDH mutations do not develop during the progression of IDH wild-type primaries and imply that IDH mutation is an early event in oncogenesis.

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

### 7. REFERENCES

The following references, to the extent that they provide exemplary procedural or other details supplementary to those set forth herein, are specifically incorporated herein by reference.
U.S. Pat. No. 3,817,837
U.S. Pat. No. 3,850,752
U.S. Pat. No. 3,939,350
U.S. Pat. No. 3,996,345
U.S. Pat. No. 4,275,149
U.S. Pat. No. 4,277,437
U.S. Pat. No. 4,366,241
U.S. Pat. No. 4,683,195
U.S. Pat. No. 4,683,202
U.S. Pat. No. 4,800,159
U.S. Pat. No. 4,883,750
U.S. Pat. No. 5,242,974
U.S. Pat. No. 5,279,721
U.S. Pat. No. 5,384,261
U.S. Pat. No. 5,405,783
U.S. Pat. No. 5,412,087
U.S. Pat. No. 5,424,186
U.S. Pat. No. 5,429,807
U.S. Pat. No. 5,436,327
U.S. Pat. No. 5,445,934
U.S. Pat. No. 5,472,672
U.S. Pat. No. 5,527,681
U.S. Pat. No. 5,529,756
U.S. Pat. No. 5,532,128
U.S. Pat. No. 5,545,531
U.S. Pat. No. 5,554,501
U.S. Pat. No. 5,556,752
U.S. Pat. No. 5,561,071
U.S. Pat. No. 5,571,639
U.S. Pat. No. 5,593,839
U.S. Pat. No. 5,599,695
U.S. Pat. No. 5,624,711
U.S. Pat. No. 5,658,734
U.S. Pat. No. 5,700,637
U.S. Pat. No. 5,757,994
U.S. Pat. No. 5,788,166
U.S. Pat. No. 5,838,002
U.S. Pat. No. 5,840,873
U.S. Pat. No. 5,843,640
U.S. Pat. No. 5,843,650
U.S. Pat. No. 5,843,651
U.S. Pat. No. 5,843,663
U.S. Pat. No. 5,846,708
U.S. Pat. No. 5,846,709
U.S. Pat. No. 5,846,717
U.S. Pat. No. 5,846,726
U.S. Pat. No. 5,846,729
U.S. Pat. No. 5,846,783
U.S. Pat. No. 5,849,481
U.S. Pat. No. 5,849,486
U.S. Pat. No. 5,849,487
U.S. Pat. No. 5,849,497
U.S. Pat. No. 5,849,546
U.S. Pat. No. 5,849,547
U.S. Pat. No. 5,851,772
U.S. Pat. No. 5,853,990
U.S. Pat. No. 5,853,992
U.S. Pat. No. 5,853,993
U.S. Pat. No. 5,856,092
U.S. Pat. No. 5,858,652
U.S. Pat. No. 5,861,244
U.S. Pat. No. 5,863,732
U.S. Pat. No. 5,863,753
U.S. Pat. No. 5,866,331
U.S. Pat. No. 5,866,366
U.S. Pat. No. 5,882,864
U.S. Pat. No. 5,900,481
U.S. Pat. No. 5,905,024
U.S. Pat. No. 5,910,407
U.S. Pat. No. 5,912,124
U.S. Pat. No. 5,912,145
U.S. Pat. No. 5,912,148
U.S. Pat. No. 5,916,776
U.S. Pat. No. 5,916,779
U.S. Pat. No. 5,919,626
U.S. Pat. No. 5,919,630
U.S. Pat. No. 5,922,574
U.S. Pat. No. 5,925,517
U.S. Pat. No. 5,928,862
U.S. Pat. No. 5,928,869
U.S. Pat. No. 5,928,905
U.S. Pat. No. 5,928,906
U.S. Pat. No. 5,929,227
U.S. Pat. No. 5,932,413
U.S. Pat. No. 5,932,451
U.S. Pat. No. 5,935,791
U.S. Pat. No. 5,935,825
U.S. Pat. No. 5,939,291
U.S. Pat. No. 5,942,391
U.S. Pat. No. 5,986,258
U.S. Pat. No. 6,004,755
U.S. Pat. No. RE 35,413
Abbondanzo, Ann. Diagn. Pathol., 3(5):318-327, 1999.
Akamatsu et al., World J. Clin. Oncol., 2:94-107, 2011.
Allred et al., Proc. Natl. Acad. Sci. USA, 87:3220-3224, 1990.
Bahr et al., J. Mass Spectrom., 32:1111-1116, 1997.
Bentzley et al., Anal Chem., 68(13):2141-2146, 1996.
Bjomsson et al., Liver, 19:501-508, 1999.
Blechacz et al., Hepatology, 48:308-321, 2008.
Bleeker et al., Hum. Mutat., 30:7-11, 2009.
Borger & Zhu, Expert Rev. Anticancer Ther. 12(5):543-546, 2012.
Brown et al. Immunol. Ser., 53:69-82, 1990.
Bucknall et al., J. Am. Soc. Mass Spectrom., 13(9):1015-1027, 2002.
Caprioli et al., Anal. Chem., 69:4751, 1997.
Chamberlan et al., In: PCR Protocols, Innis et al. (Eds.), Academic Press, NY, 272-281, 1990.
Chaurand et al., Anal Chem., 71(23):5263-5270, 1999.
Chen et al., Biomed Chromatogr., 15(8):518-24, 2001.
Dang et al., Nature, 462:739-744, 2009.
De Jager et al., Semin. Nucl. Med., 23(2):165-179, 1993.
Desiderio et al., J. Mass Spectrom., 35(6):725-733, 2000.
Desiderio et al., Methods Mol. Biol., 61:57-65, 1996.
Doolittle and Ben-Zeev, Methods Mol, Biol., 109:215-237, 1999.
Duncan et al., Rapid Commun. Mass Spectrom., 7(12):1090-1094, 1993.
European Appln. 0 364 255
European Appln. 320 308
European Appln. 329 822
Faulstich et al., Anal. Chem., 69(21):4349-4353, 1997.
Fenn et al., Science, 246(4926):64-71, 1989.
Frohman, In: PCR Protocols: A Guide To Methods And Applications, Academic Press, N.Y., 1990.
Gatto et al., World J. Gastrointest. Oncol., 2:136-45, 2010. GB Appln. 2 202 328
Gobom et al., Anal. Chem., 72(14):3320-3326, 2000.
Gulbis and Galand, Hum. Pathol., 24(12):1271-1285, 1993.
Hartmann et al., Acta Neuropathol., 118:469-474, 2009.
Hartmann et al., Acta. Neuropathol., 118:469-474, 2009.
Horak et al., Rapid Commun. Mass Spectrom., 15(4):241-248, 2001.
Ichimura et al., Neuro. Oncol., 11:341-347, 2009.
Innis et al., Proc. Natl. Acad. Sci. USA, 85(24):9436-9440, 1988.
Jespersen et al., Anal Chem., 71(3):660-666, 1999.
Jiang et al., Biochem. Pharmacol., 59:763-772, 2000.
Kabarle et al., Anal. Chem. 65(20):972A-986A, 1993.
Kanazawa et al., Biol. Pharm. Bull., 22(4):339-346, 1999.
Kazmaier et al., Anesthesiology, 89(4):831-817, 1998.
Kwoh et al., Proc. Natl. Acad. Sci. USA, 86:1173, 1989.
Lazaridis et al., Gastroenterology, 128:1655-1667, 2005.
Levy et al., Dig. Dis. Sci., 50:1734-1740, 2005.
Li et al., J. Biol. Chem., 275:29823-29828, 2000.
Lovelace et al., J. Chromatogr., 562(1-2):573-584, 1991.
Lynn et al., J. Mol. Evol., 48(5):605-614, 1999.
Marie et al., Anal. Chem., 72(20):5106-5114, 2000
Miketova et al., Mol. Biotechnol., 8(3):249-253, 1997.
Mirgorodskaya et al., Rapid Commun. Mass Spectrom., 14(14):1226-1232, 2000.
Muddiman et al., Fres. J. Anal. Chem., 354:103, 1996.
Mueller and Wold, Science 246, 780-786, 1989.
Nakamura et al., In: Handbook of Experimental Immunology (4^{th} Ed.), Weir et al. (Eds), 1:27, Blackwell Scientific Publ., Oxford, 1987.
Nehls et al., Semin. Liver Dis., 24:139-154, 2004.
Nelson et al., Rapid Commun. Mass Spectrom., 8(8):627-631, 1994.
Nguyen et al., J. Chromatogr. A., 705(1):21-45, 1995.
Ohara et al., Proc. Natl. Acad. Sci. USA, 86:5673-5677, 1989.
Patel et al., Nat. Rev. Gastroenterol. Hepatol., 8:189-200, 2011.
PCT Appln. PCT/US87/00880
PCT Appln. PCT/US89/01025
PCT Appln. WO 88/10315
PCT Appln. WO 89/06700
PCT Appln. WO 90/07641
Pietrak et al., Biochemistry, 50:4804-4812, 2011.
Ramage et al., Gastroenterology, 108:865-869, 1995.
Reitman et al., Cancer Cell, 17:215-216, 2010.
Reitman et al., Proc. Natl. Acad. Sci. USA, 108:3270-3275, 2011.
Roepstorff, In: MALDI-TOF Mass Spectrometry Protein Chemistry, Jolles and Jörnvall (Eds.), 1-220, 2000.
Rosen et al., Transpl. Int., 23:692-697, 2010.
Sambrook and Russell, Molecular Cloning: A Laboratory Manual, 3rd Edition, Cold Spring Harbor Laboratory, N.Y., 1989.
Sanson et al., J. Clin. Oncol., 27:4150-4154, 2009.
Stoeckli et al., Nat. Med., 7(4):493-496, 2001.
Takach et al., J. Protein Chem., 16:363, 1997.
Tefferi et al., Leukemia, 24:1302-1309, 2010.
Villanueva et al., Genes Dev., 13:3160-3169, 1999.
Walker et al., Proc. Natl. Acad. Sci. USA, 89:392-396, 1992.
Walker, PCR Meth. Appl., 3:1-6, 1993.
Wang et al., J. Am. Soc. Mass Spectrom., 10(4):329-338, 1999.
Ward et al., Cancer Cell, 17:225-234, 2010.
Watanabe et al., Am. J. Pathol., 174:1149-1153, 2009.
Wittmann et al., Biotechnol. Bioeng., 72:642, 2001.
Wu et al., Anal. Biochem., 263(2):129-38, 1998.
Wu et al., Rapid Commun Mass Spectrom., 14(9):756-64, 2000.
Yan et al., N. Engl. J. Med., 360:765-773, 2009.
Yang et al., J. Agric. Food Chem., 48(9):3990-6, 2000.
Zhong et al., Clin. Chem. ACTA., 313:147, 2001.
Zweigenbaum et al., Anal. Chem., 71(13):2294-300, 1999.
Zweigenbaum et al., J. Pharm. Biomed. Anal., 23(4):723-733, 2000.

What is claimed is:

1. A method of diagnosing a cholangiocarcinoma tumor of intrahepatic origin in a mammal comprising:
(a) performing a histologic analysis of a tumor cell-containing sample from said mammal, whereby glioma and secondary glioblastomas, acute myeloid leukemia, and chondrosarcoma are excluded by histology; and
(b) sequencing a isocitrate dehydrogenase 1 (IDH1) or isocitrate dehydrogenase 2 (IDH2) encoding polynucleotide in from a tumor-cell containing sample from said mammal to identify the presence or absence of a mutation in IDH1 or IDH2, wherein the presence of a mutation in IDH1 or IDH2 excludes distal extrahepatic cholangiocarcinoma,
thereby diagnosing a cholangiocarcinoma of intrahepatic origin.

2. The method of claim 1, wherein the mutation is R132c in IDH1.

3. The method of claim 1, wherein the mutation is R132S in IDH1.

4. The method of claim 1, wherein the mutation is R132G in IDH1.

5. The method of claim 1, wherein the mutation is R132L in IDH1.

6. The method of claim 1, wherein the mutation is R172M in IDH2.

7. The method of claim 1, wherein the mutation is R172K in IDH2.

8. The method of claim 1, wherein the mutation is R172G in IDH2.

9. The method of claim 1, wherein the mammal is a human.

10. The method of claim 1, further comprising measuring 2-hydroxyglutarate in a tumor from said mammal.

11. A method of treating a cholangiocarcinoma tumor in a mammal comprising:
(a) sequencing a isocitrate dehydrogenase 1 (IDH1) or isocitrate dehydrogenase 2 (IDH2) encoding polynucleotide in from tumor sample from said mammal to identify the presence or absence of a mutation in IDH1 or IDH2;
(b) treating said mammal with an inhibitor of 2-hydroxyglutarate synthesis or function when a mutation in IDH1 or IDH2 is found.

12. The method of claim 11, wherein the mutation is R132c in IDH1.

13. The method of claim 11, wherein the mutation is R132S in IDH1.

14. The method of claim 11, wherein the mutation is R132G in IDH1.

15. The method of claim 11, wherein the mutation is R132L in IDH1.

16. The method of claim 11, wherein the mutation is R172M in IDH2.

17. The method of claim 11, wherein the mutation is R172K in IDH2.

18. The method of claim 11, wherein the mutation is R172G in IDH2.

19. The method of claim 11, wherein the mammal is a human.

20. The method of claim 11, further comprising measuring 2-hydroxyglutarate in a tumor from said mammal.

21. A method for predicting the survival of a mammal having a cholangiocarcinoma tumor comprising sequencing a isocitrate dehydrogenase 1 (IDH1) or isocitrate dehydrogenase 2 (IDH2) encoding polynucleotide in from tumor sample from said mammal to identify the presence or absence of a mutation in IDH1 or IDH2, whereby the presence of IDH1 and/or IDH2 mutation indicates better overall survival than the absence of IDH1 and/or IDH2 mutation.

22. The method of claim 21, wherein the mutation is R132c in IDH1.

23. The method of claim 21, wherein the mutation is R132S in IDH1.

24. The method of claim 21, wherein the mutation is R132G in IDH1.

25. The method of claim 21, wherein the mutation is R132L in IDH1.

26. The method of claim 21, wherein the mutation is R172M in IDH2.

27. The method of claim 21, wherein the mutation is R172K in IDH2.

28. The method of claim 21, wherein the mutation is R172G in IDH2.

29. The method of claim 21, wherein the mammal is a human.

30. The method of claim 21, further comprising measuring 2-hydroxyglutarate in a tumor from said mammal.
